(19) United States
(12) Patent Application Publication
(10) Pub. No.: US 2026/0232326 A1
(43) Pub. Date: Aug. 13, 2026

(54) PATIENT-SPECIFIC GUIDES, SYSTEMS, AND METHODS

(71) Applicant: CATALYST ORTHOSCIENCE INC., Naples, FL (US)

(72) Inventors: Logan MORTON, Gainesville, FL (US); Ephraim AKYUZ, Salt Lake City, UT (US); Theodore BLAINE, Old Greenwich, CT (US)

(21) Appl. No.: 19/537,333

(22) Filed: Feb. 11, 2026

Related U.S. Application Data
(60) Provisional application No. 63/756,868, filed on Feb. 11, 2025.

(51) Int. Cl. A61B 17/15; A61B 34/10

(52) U.S. Cl. CPC A61B 17/155; A61B 34/10

(57) ABSTRACT

An orthopedic guide may include a guide body configured to couple with a bone of a patient, a guide feature associated with the guide body configured to guide a surgical instrument relative to the bone, and a bone-facing surface associated with the guide body comprising a patient-specific surface formed thereon and corresponding to an anatomical feature of the bone. The patient-specific surface may be configured to position the guide feature at a desired orientation relative to the bone as the patient-specific surface engages the anatomical feature on the bone.

### CROSS-REFERENCE TO RELATED APPLICATIONS

This application claims the benefit of U.S. Provisional Patent Application Serial No. 63/756,868 filed on February 11, 2025, entitled “PATIENT-SPECIFIC INSTRUMENT GUIDES”. The above-referenced document is hereby incorporated by reference in its entirety.

### TECHNICAL FIELD

The present disclosure relates to orthopedic devices, systems, and methods. More specifically, the present disclosure relates to patient-specific orthopedic guides, systems, and methods for guiding surgical instruments relative to a bone of a patient.

### BACKGROUND

Joint arthroplasty procedures are conducted to restore the function of an unhealthy joint. Typically, these procedures involve replacing the damaged natural articular surfaces of the joint with artificial articular surfaces to restore joint function. The new joint implants are typically anchored into adjacent bones of the joint to maintain long term stability.

In shoulder arthroplasty, a humeral implant is attached to the humerus and a glenoid implant is attached to the glenoid area on the scapula. There are two major types of shoulder arthroplasty procedures: anatomic and reverse. In an anatomic procedure, the implants are intended to replicate existing anatomy, where the humeral head is replaced with a similarly shaped convex hemispherical articular surface (i.e., a ball or glenosphere-shaped implant), and the glenoid is replaced with a concave articular surface (i.e., a concave-shaped socket implant). In a reverse shoulder arthroplasty procedure, these ball and socket structures are reversed such that the concave articular surface is affixed to the humerus and the convex articular surface is affixed to the glenoid.

The humerus and glenoid are typically prepared and shaped via one or more surgical instruments before the implants are received thereon to ensure their proper locations/orientations and achieve proper joint function. However, planning and executing these bone-shaping surgical steps can be difficult because the bone and joint morphologies for each individual patient can vary drastically. Thus, patient-specific orthopedic guides, systems, and methods that can guide surgical instruments relative to specific bones of a patient would be desirable.

Although the patient-specific guides, systems, and methods of the present disclosure are described herein with respect to shoulder arthroplasty procedures, it will be understood that the patient-specific guides, systems, and methods described herein can be adapted to any orthopedic surgical procedure or application within a patient including, but not limited to surgical procedures or applications within the: hip, knee, elbow, ankle, foot, wrist, hand, spine, etc.

### SUMMARY

The patient-specific guides, systems, and methods of the present disclosure have been developed in response to the present state of the art, and in particular, in response to the problems and needs in the art that have not yet been fully solved by currently available patient-specific guides, systems, and methods. In some embodiments, the patient-specific guides, systems, and methods of the present disclosure may provide improved guides, systems, and methods for guiding a surgical instrument relative to a bone of a patient.

In some embodiments, an orthopedic guide may include a guide body configured to couple with a humerus of a patient, at least two guide features associated with the guide body that are configured to guide at least one surgical instrument relative to the humerus, and a bone-facing surface associated with the guide body comprising a patient-specific surface formed thereon corresponding to an anatomical feature of the humerus. The patient-specific surface may be configured to position the at least two guide features along at least two desired orientations relative to the humerus as the patient-specific surface engages the anatomical feature on the humerus.

The orthopedic guide according to any preceding paragraph, wherein that at least one surgical instrument may include a bone cutting instrument, and the at least two guide features may include a first cutting guide feature that may be configured to guide the bone cutting instrument into the humerus along a first desired plane that may correspond to a first implant surface of an implant that may be configured to be installed on a first prepared surface of the humerus that is formed along the first desired plane, as well as a second cutting guide feature that may be configured to guide the bone cutting instrument into the humerus along a second desired plane that may correspond to a second implant surface of the implant that may be configured to be installed on a second prepared surface of the humerus that is formed along the second desired plane.

The orthopedic guide according to any preceding paragraph, wherein at least one of the first desired plane and the second desired plane is not parallel to an anatomic neck plane of the humerus.

The orthopedic guide according to any preceding paragraph, wherein the at least one surgical instrument may also include an elongate surgical instrument, and the at least two guide features may also include a reference guide cannula configured to guide the elongate surgical instrument into the humerus along a desired axis.

The orthopedic guide according to any preceding paragraph, wherein the orthopedic guide may also include at least one guide arm extending from the guide body with a patient-specific surface formed thereon.

The orthopedic guide according to any preceding paragraph, wherein the at least one guide arm may be configured to selectively break away from the guide body.

The orthopedic guide according to any preceding paragraph, wherein the patient-specific surface may be at least partially formed via an additive manufacturing process.

The orthopedic guide according to any preceding paragraph, wherein the orthopedic guide may also include an orientation feature configured to indicate an orientation of the orthopedic guide relative to the humerus.

In some embodiments, an orthopedic guide may include a guide body configured to couple with a humerus of a patient, a cutting guide feature coupled with the guide body and configured to guide a cutting instrument along a desired cutting plane relative to the humerus that is not parallel to an anatomic neck plane of the humerus, a first patient-specific surface formed on a first bone-facing surface of the guide body that corresponds to a first anatomical feature of the humerus, and at least one guide arm coupled to and extending away from the guide body, the at least one guide arm including a second patient-specific surface formed on a second bone-facing surface of the at least one guide arm that corresponds to a second anatomical feature of the humerus. The cutting guide feature may be positioned along the desired cutting plane relative to the humerus when the first patient-specific surface engages the first anatomical feature of the humerus, and the second patient-specific surface engages the second anatomical feature of the humerus.

The orthopedic guide according to any preceding paragraph, wherein the cutting guide feature may include a cutting guide surface.

The orthopedic guide according to any preceding paragraph, wherein the cutting guide feature may include a cutting guide slot.

The orthopedic guide according to any preceding paragraph, wherein the orthopedic guide may also include a guide body projection coupled to and extending away from the guide body.

The orthopedic guide according to any preceding paragraph, wherein the at least one guide arm may include a guide arm bud coupled thereto that may include the second patient-specific surface formed thereon.

The orthopedic guide according to any preceding paragraph, wherein the at least one guide arm may be configured to selectively break away from the guide body.

The orthopedic guide according to any preceding paragraph, wherein the orthopedic guide may also include a fixation guide configured to receive a securement pin therethrough to couple the orthopedic guide to the humerus.

In some embodiments, an orthopedic guide may include a first guide body configured to couple with a bone of a patient that may include a first cutting guide feature, a second guide body configured to couple with the bone of the patient that may include a second cutting guide feature, at least one guide body bridge coupled intermediate the first guide body and the second guide body, and at least one bone-facing surface associated with the orthopedic guide that may include at least one patient-specific surface formed thereon that corresponds to at least one anatomical feature of the bone. When the at least one patient-specific surface is engaged with the at least one anatomical feature of the bone: the first cutting guide feature may be positioned at a first desired orientation relative to the bone, and the second cutting guide feature may be positioned at a second desired orientation relative to the bone.

The orthopedic guide according to any preceding paragraph, wherein the first cutting guide feature may include a first cutting surface encapsulated within a first cutting guide slot that may be configured to guide a cutting surgical instrument into the bone along a first desired plane, and the second cutting guide feature may include a second cutting surface encapsulated within a second cutting guide slot that may be configured to guide the cutting surgical instrument into the bone along a second desired plane.

The orthopedic guide according to any preceding paragraph, wherein at least one of the first guide body and the second guide body may include a lattice structure.

The orthopedic guide according to any preceding paragraph, wherein the orthopedic guide may also include at least one guide arm extending from the orthopedic guide that may include the at least one patient-specific surface formed thereon.

The orthopedic guide according to any preceding paragraph, wherein the orthopedic guide may also include a reference guide cannula configured to guide an elongate surgical instrument into the bone along a desired axis.

The orthopedic guide according to any preceding paragraph, wherein the bone may be a humerus of the patient.

The orthopedic guide according to any preceding paragraph, wherein the orthopedic guide may also include at least one fixation guide configured to receive at least one securement pin therethrough to secure the orthopedic guide to the bone.

These and other features and advantages of the present disclosure will become more fully apparent from the following description and appended claims or may be learned by the practice of the patient-specific guides, systems, and methods set forth hereinafter.

### BRIEF DESCRIPTION OF THE DRAWINGS

Exemplary embodiments of the present disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the present disclosure will be described with additional specificity and detail through use of the accompanying drawings in which:

FIG. 1A is a perspective view of an orthopedic guide, according to an embodiment of the present disclosure;

FIG. 1B is another perspective view of the orthopedic guide of FIG. 1A;

FIG. 1C is another perspective view of the orthopedic guide of FIG. 1A;

FIG. 1D is another view of the orthopedic guide shown in FIG. 1C illustrating textured surfaces on the orthopedic guide;

FIG. 2 is a perspective side view of the orthopedic guide of FIG. 1A engaged with a humeral head;

FIG. 3 is a perspective side view of the orthopedic guide of FIG. 1A coupled to the humeral head with securement pins;

FIG. 4 is a perspective side view of an orthopedic guide, according to another embodiment of the present disclosure;

FIG. 5 is a perspective side view of the orthopedic guide of FIG. 4 with the guide arm removed;

FIG. 6 is a perspective view of a resected humeral head with a first prepared surface;

FIG. 7 is a perspective view of the resected humeral head of FIG. 6 with a first implant adapter coupled thereto;

FIG. 8 is a perspective view of the resected humeral head of FIG. 6 with a bone cavity formed therein;

FIG. 9 is a perspective view of the resected humeral head shown in FIG. 8 with a second implant adapter coupled therein;

FIG. 10A is a perspective front view of an orthopedic guide, according to another embodiment of the present disclosure;

FIG. 10B is a perspective side view of the orthopedic guide of FIG. 10A;

FIG. 10C is a perspective bottom view of the orthopedic guide of FIG. 10A;

FIG. 10D is another perspective bottom view of the orthopedic guide of FIG. 10A;

FIG. 11 is a perspective side view of humeral head before resection;

FIG. 12 illustrates the humeral head of FIG. 11 with the orthopedic guide of FIG. 10A engaged therewith;

FIG. 13 illustrates the humeral head of FIG. 12 with securement pins coupling the orthopedic guide thereto;

FIG. 14 is a perspective side view of an orthopedic guide coupled to a humeral head, according to another embodiment of the present disclosure;

FIG. 15 is a perspective side view of an orthopedic guide coupled to a humeral head, according to another embodiment of the present disclosure;

FIG. 16A is a perspective view of an orthopedic guide coupled to a humeral head, according to another embodiment of the present disclosure;

FIG. 16B is another perspective view of the orthopedic guide of FIG. 16A;

FIG. 16C is another perspective view of the orthopedic guide of FIG. 16A;

FIG. 16D is another perspective view of the orthopedic guide of FIG. 16A;

FIG. 17A is a perspective view of an orthopedic guide, according to another embodiment of the present disclosure;

FIG. 17B is another perspective view of the orthopedic guide of FIG. 17A;

FIG. 17C is another perspective view of the orthopedic guide of FIG. 17A;

FIG. 17D is another perspective view of the orthopedic guide of FIG. 17A;

FIG. 18 is another perspective view of the orthopedic guide of FIG. 17A coupled to a humeral head;

FIG. 19 is another perspective view of the orthopedic guide of FIG. 17A coupled to a humeral head;

FIG. 20 is a perspective view of a resected humeral head with first and second prepared surfaces;

FIG. 21 is a perspective side view of an orthopedic guide coupled to a humeral head, according to another embodiment of the present disclosure;

FIG. 22A is a perspective front view of an orthopedic guide, according to another embodiment of the present disclosure;

FIG. 22B is a perspective side view of the orthopedic guide of FIG. 22A;

FIG. 22C is a perspective bottom view of the orthopedic guide of FIG. 22A;

FIG. 22D is another perspective bottom view of the orthopedic guide of FIG. 22A;

FIG. 23 is a perspective side view of a humeral head with the orthopedic guide of FIG. 22A engaged therewith;

FIG. 24 illustrates the humeral head and orthopedic guide shown in FIG. 23 with securement pins coupling the orthopedic guide thereto;

FIG. 25 is a perspective view of a resected humeral head with first, second, third, and fourth prepared surfaces and a humeral implant engaging thereon;

FIG. 26A is a perspective side view of an orthopedic guide, according to another embodiment of the present disclosure;

FIG. 26B is a perspective distal end view of the orthopedic guide of FIG. 26A;

FIG. 26C is a perspective proximal end view of the orthopedic guide of FIG. 26A;

FIG. 27 is a perspective proximal end view of the orthopedic guide of FIG. 26A coupled to a glenoid on a scapula bone;

FIG. 28 is a perspective proximal end view of an orthopedic guide coupled to a glenoid on a scapula bone, according to another embodiment of the present disclosure;

FIG. 29 is a perspective proximal end view of an orthopedic guide coupled to a glenoid on a scapula bone, according to another embodiment of the present disclosure;

FIG. 30 is a perspective proximal end view of an orthopedic guide coupled to a glenoid on a scapula bone, according to another embodiment of the present disclosure;

FIG. 31 is a perspective side view of a scapula bone and a drill bit oriented to penetrate the glenoid cavity along a desired trajectory;

FIG. 32 is a perspective side view of the scapula bone of FIG. 31 showing a reference bone tunnel formed in the glenoid cavity along the desired trajectory; and

FIG. 33 is a perspective side view of the glenoid cavity of FIG. 32 prepared to receive a glenoid implant thereon.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word "exemplary" is used herein to mean "serving as an example, instance, or illustration." Any embodiment described herein as "exemplary" is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms may also be applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means outboard deviation of the knees (away from the sagittal plane) from the line between the hip and ankle, resulting in a “bowlegged” stance. Valgus means inboard deviation of the knees (toward the sagittal plane) from the line between the hip and ankle, resulting in a “knock-kneed” stance.

As used herein, the term patient-specific surface, area, planar area, line, or point can comprise any possible shape(s) that may correspond to patient-specific anatomical feature(s) including, but not limited to: one or more one dimensional shapes (e.g., a point, such as a patient-specific point that references a specific anatomical feature corresponding to the point), one or more two dimensional shapes (e.g., a line or planar area, such as a patient-specific line or planar area that references a specific anatomical feature corresponding to the line or planar area), and one or more three dimensional shapes (e.g., a surface area comprising a three-dimensional shape), or any combinations thereof.

It will be understood that any orthopedic guide described or contemplated herein may (or may not) include any feature, shape, or morphology that is described or contemplated herein with respect to any orthopedic guide that is described or contemplated herein. Moreover, any of the orthopedic guides and/or surgical instruments that are described or contemplated herein may be combined in any manner to produce any number of different surgical kits.

The arthroplasty procedures described or contemplated herein can be planned preoperatively with planning software to analyze preoperative medical images (e.g., MRI’s, CT scans, X-rays, ultrasounds, etc.) of a patient’s unique anatomical features to replicate the bone structures of the patient in a virtual three-dimensional (3D) environment. This allows the surgeon to visualize the patient’s existing morphology, plan/simulate how the surgical operation can be conducted, and identify the desired final locations for the implant(s). The software can also allow for the creation of patient-specific instrumentation that can be utilized to help guide the placement of surgical instruments and/or implants during the surgical procedure via patient-specific surfaces formed on the orthopedic guides that are described or contemplated herein to interface with specific anatomical features of an individual patient. In this manner, patient-specific orthopedic guides can be manufactured (e.g., via an additive manufacturing process, etc.) to incorporate these one or more patient-specific surfaces on the orthopedic guides that correspond to one or more anatomical features on the bone(s) of the patient. The orthopedic guides can be manufactured from biocompatible plastics and/or metals including, but not limited to: ABS, Nylon, Polyamide, titanium, stainless steel, etc.

The patient-specific portions or surfaces formed on the orthopedic guide(s) during manufacture can assume the “negative” shapes of one or more specific anatomical landmarks or features on the patient’s bone including, but not limited to: the greater tuberosity, the lesser tuberosity, the bicipital groove, the anatomic neck, an articular surface of the humeral head, any part of the scapular anatomy such as the glenoid surface, the glenoid rim, the infraglenoid tubercle, the scapular neck, the coracoid, the acromion, etc. In this manner, the patient-specific portions or surfaces formed on the orthopedic guide can act to position the orthopedic guide at a desired orientation relative to the bone as the patient-specific surfaces engage their corresponding anatomical features on the bone. The patient-specific surfaces of the guide can interface with the bony landmarks to orient the guide toward a single orientation relative to the bone such that the guide may “automatically assume” its proper orientation relative to the bone once these complementary-shaped surfaces intermesh with each other.

In some embodiments, the main body of the guide may also include guide arms extending therefrom with bone interfacing features formed on their ends configured to reference additional bony landmarks. In some embodiments, the patient-specific surfaces can mate or interface with the patient’s bony features/landmarks in a strong/specific manner that the guide may appear to “actively grab” and/or lock itself to the patient’s bone. This behavior will simplify the surgical procedure and ensure that the guide(s) can only be placed in one proper orientation relative to the bone such that all subsequent bone preparation steps will proceed in their proper orientation(s).

In this manner, one or more guide features associated with the guide body can be configured to guide one or more surgical instruments relative to the bone along one or more desired orientations. For example, the at least one guide feature may include at least one cutting surface that can be utilized to guide a saw blade or other cutting tool to remove a specific section of bone along a specific/desired cutting plane. The cutting surface can be a flat surface for a saw blade to engage and “sit flat” against, or a cutting guide slot that may receive the saw blade therein and guide the trajectory of the saw blade along a specific/desired cutting plane.

As another example, the at least one guide feature may include a central cannulation or reference guide cannula that may align with a patient-specific axis determined preoperatively to guide an elongate surgical instrument (e.g., a pin, drill, guide wire, K-wire, etc.) along the patient-specific axis and into the bone. Once the elongate surgical instrument is inserted into the bone along the patient-specific axis, the elongate surgical instrument may then be utilized to guide another surgical instrument (or an implant) relative to the bone 10 to a desired location on or within the bone. For example, the elongate surgical instrument may be utilized to guide a cannulated reamer (not shown) towards a surface of the bone 10 during a reaming operation that may shape the bone to achieve a desired shape and/or remove a specific amount of the bone during the reaming operation.

In another example, a drill may be utilized to create a hole or series of holes that may create one or more interface points for subsequent surgical tools (or an implant) during the procedure.

In another example, other cannulated portions or guides on the guide can serve as drill guide holes that may direct the surgeon to drill a series of holes or bone tunnels in the bone for use in subsequent steps of a procedure (e.g., such as subscapularis tendon repair with sutures, suture anchors, suture needles passing through the bone tunnels, etc.). One non-limiting example of such an application may include guiding the surgeon to drill one or more holes on the medial side of the lesser tuberosity of a humeral bone, as well as one or more holes on the lateral side of the lesser tuberosity. These drill holes may approximate or intersect with each other at some point such that one or more bone tunnels may be created from the medial side to the lateral side of the lesser tuberosity. Other bone tunnels can also be created through other locations on the humerus and/or blind holes may also be created that do not fully penetrate through the humerus, etc.

In some embodiments, the guide can have one or more reference guide cannulas and/or one or more fixation guides/tubes/conduits that may be utilized to guide one or more securement pins, fixation elements, screws, drill bits, etc., therethrough and into the bone to secure the guide to the bone. The bores of the reference guide cannula(s) and/or the fixation guides/tubes/conduits can be reinforced with metal inserts (e.g., stainless steel, titanium, etc.) to reinforce them and/or help them resist abrasive wear during the procedure.

In some instances, the guides or portions of the guides may include one or more webbed or lattice structures/geometries to reduce an amount of material utilized during an additive manufacturing process and reduce cost.

In other instances, the design of the guide may incorporate one or more portions that can be manually broken off during use by the surgeon. These breakaway sections may allow the guide to be utilized sequentially to perform different steps of the procedure. For example, the guide may be utilized to perform an earlier step of the surgical procedure relative to the bone, after which a portion of the guide can be manually broken off (or otherwise removed from the guide) to allow the surgeon to perform a subsequent step in the surgical procedure relative to a different portion of the guide that is revealed. In one non-limiting example, a first section of the guide may allow for a drill to create a location-specific hole in the bone, after which the first section of the guide may be broken off to expose a second section of the guide that may be utilized to cut a portion of the bone with a saw, and then the second section may be broken off to expose a third portion of the guide that may be utilized for a different portion of the surgical procedure, etc.

In some embodiments, the guides disclosed or contemplated herein may also be manufactured/printed to include labeling, instructions for use, and/or any other patient identifier information thereon to help guide/inform the surgeon during a procedure.

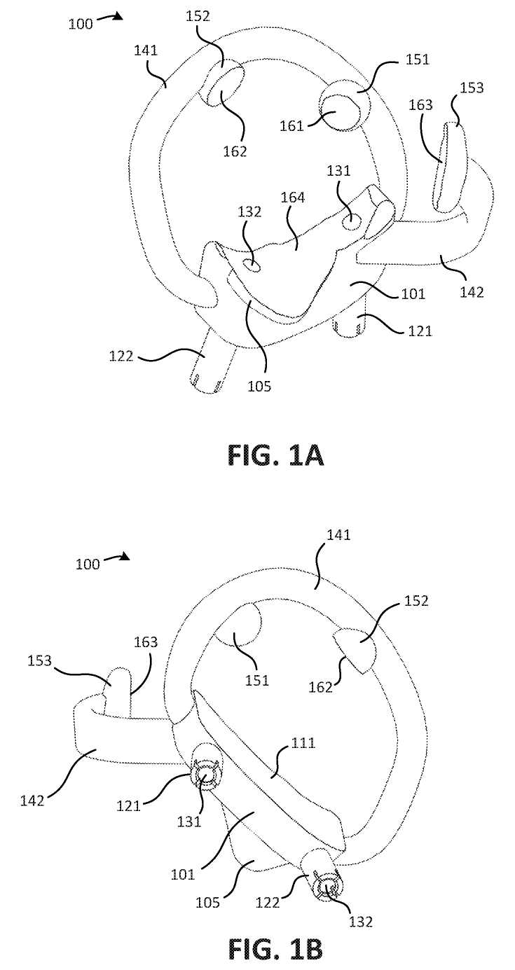

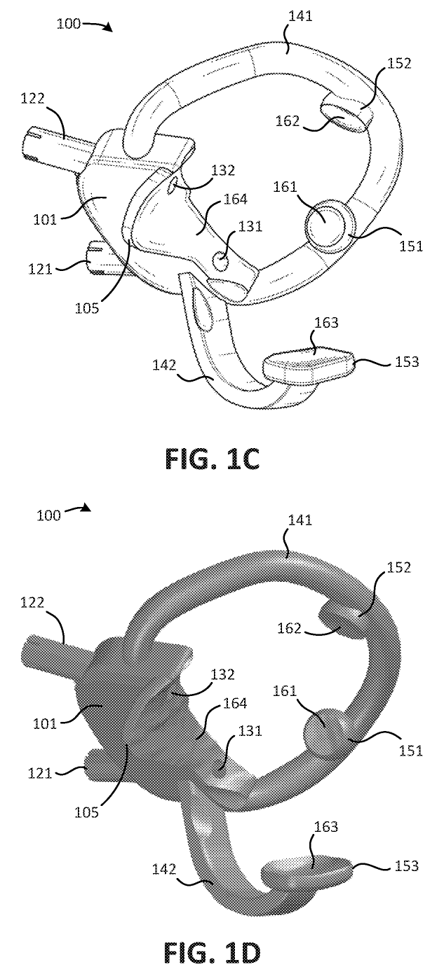

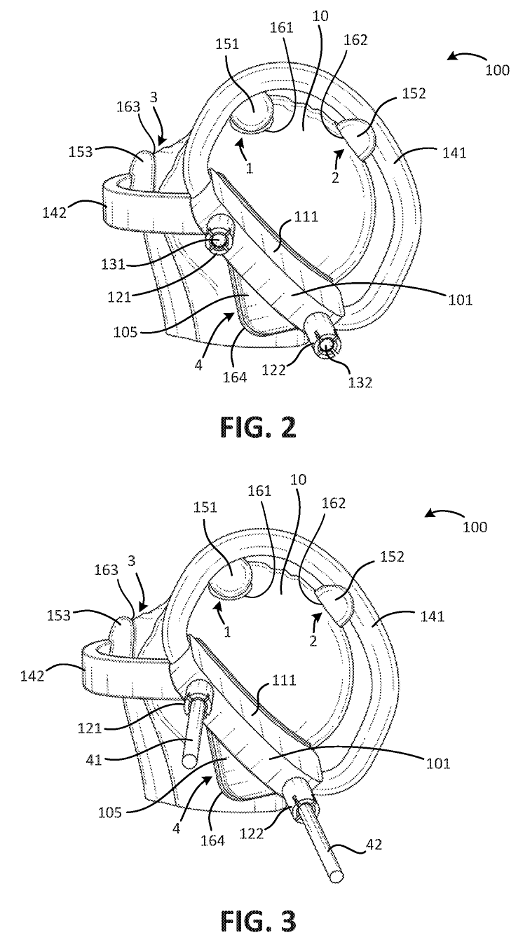

FIGS. 1A-3 illustrate an orthopedic guide 100, according to an embodiment of the present disclosure. Specifically, FIGS. 1A-1D show various perspective views of the orthopedic guide 100, FIG. 2 shows the orthopedic guide 100 engaged with a bone 10 (e.g., a humeral head, etc.), and FIG. 3 shows the orthopedic guide 100 securely pinned to the bone 10 via a first securement pin 41 and a second securement pin 42 that may be inserted through the orthopedic guide 100 and into the bone 10.

In some embodiments, the orthopedic guide 100 may generally include: a guide body 101; a cutting guide surface 111 on a superior surface of the guide body 101; a guide body projection 105 that may project away from a bone-facing surface and/or an inferior surface of the guide body 101; a first fixation guide 121 coupled to a proximal surface of the guide body 101 that may include a first fixation guide bore 131 extending through the guide body 101; a second fixation guide 122 coupled to the proximal surface of the guide body 101 that may include a second fixation guide bore 132 extending through the guide body 101; a first guide arm 141 extending from a first side surface of the guide body 101 to a second side surface of the guide body 101 along a first curved path; a second guide arm 142 extending from the inferior surface of the guide body 101 distally along a second curved path; a first guide arm bud 151 and/or a second guide arm bud 152 coupled to the first guide arm 141; a third guide arm bud 153 coupled to the second guide arm 142; a first patient-specific surface 161 formed on the first guide arm bud 151; a second patient-specific surface 162 formed on the second guide arm bud 152; a third patient-specific surface 163 formed on the third guide arm bud 153; and a fourth patient-specific surface 164 formed on the bone-facing surface of the guide body 101 and/or on the guide body projection 105 (if present).

However, it will be understood that in other embodiments the orthopedic guide 100 may include any number of guide bodies, cutting guide surfaces/slots, guide body projections, fixation guides/bores, reference guide cannulas, guide arms, guide arm buds, patient-specific surfaces, etc., and/or may incorporate any other feature, morphology, shape, or orientation that is disclosed or contemplated herein with respect to any other guide.

As best shown in FIG. 1D, each of the patient-specific surfaces (161, 162, 163, 164) formed on the orthopedic guide 100 can be shaped to correspond to specific anatomical features or areas on the bone of the patient to help position the orthopedic guide 100 at a predetermined/desired location or orientation relative to the bone 10 when each of these patient-specific surfaces (161, 162, 163, 164) engage with their corresponding anatomical feature on the bone 10. As one non-limiting example, FIGS. 2 and 3 show how the first patient-specific surface 161 can engage against a first anatomical feature 1 on the bone 10, the second patient-specific surface 162 can engage against a second anatomical feature 2 on the bone 10, the third patient-specific surface 163 can engage against a third anatomical feature 3 on the bone 10, and the fourth patient-specific surface 164 can engage against a fourth anatomical feature 4 on the bone 10. This will prompt the orthopedic guide 100 to “self-locate” into its proper orientation relative to the bone 10 and couple the guide body 101 to the bone 10 with at least one guide feature associated with the guide body 101 positioned at a desired orientation relative to the bone 10, as the patient-specific surfaces (161, 162, 163, 164) engage their corresponding anatomical features (1, 2, 3, 4).

In some embodiments, the patient-specific surfaces (161, 162, 163, 164) and/or any other portion of the orthopedic guide 100 can be at least partially formed by an additive manufacturing process. However, it will be understood that the patient-specific surfaces (and/or any other portion of any other guide that is described or contemplated herein) may be formed by any other manufacturing process.

In some embodiments, at least one guide feature associated with the guide body 101 may comprise a reference guide cannula (e.g., see the reference guide cannula 922 shown in FIG. 21, as one non-limiting example), which may be configured to guide an elongate surgical instrument therethrough (e.g., a guide wire, K-wire, drill bit, reference pin, etc.) and into the bone 10 along a desired axis (e.g., a patient-specific axis that may be determined preoperatively, etc.).

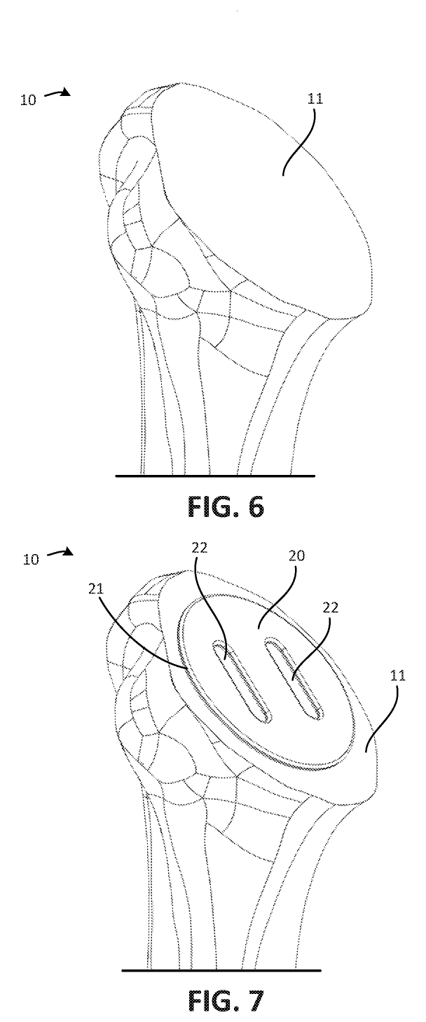

In some embodiments, the at least one guide feature may comprise a cutting guide surface, such as the cutting guide surface 111 disposed on the superior surface of the guide body 101 and configured to guide a surgical instrument (e.g., a saw blade, etc.) into the bone 10 along a desired cutting plane trajectory in order to resect a portion of the bone 10 and create a prepared surface on the bone (e.g., see the first prepared surface 11 on the bone 10 in FIG. 6).

In some embodiments, the at least one guide feature may include a cutting guide slot formed through the guide body 101 (e.g., see the cutting guide slot 311 in FIG. 5), which may be configured to guide a surgical instrument (e.g., a saw blade, etc.) into the bone 10 along a desired cutting plane trajectory in order to resect a portion of the bone 10 and create a prepared surface on the bone (e.g., see FIG. 6).

In some embodiments, the orthopedic guide 100 may also include one or more markings that may be printed or otherwise formed on the guide including, but not limited to: one or more orientation features configured to indicate an orientation of the orthopedic guide 100 relative to the bone 10 of the patient, any instructions for use during the surgical procedure, any patient identifier information, any other labeling, etc.

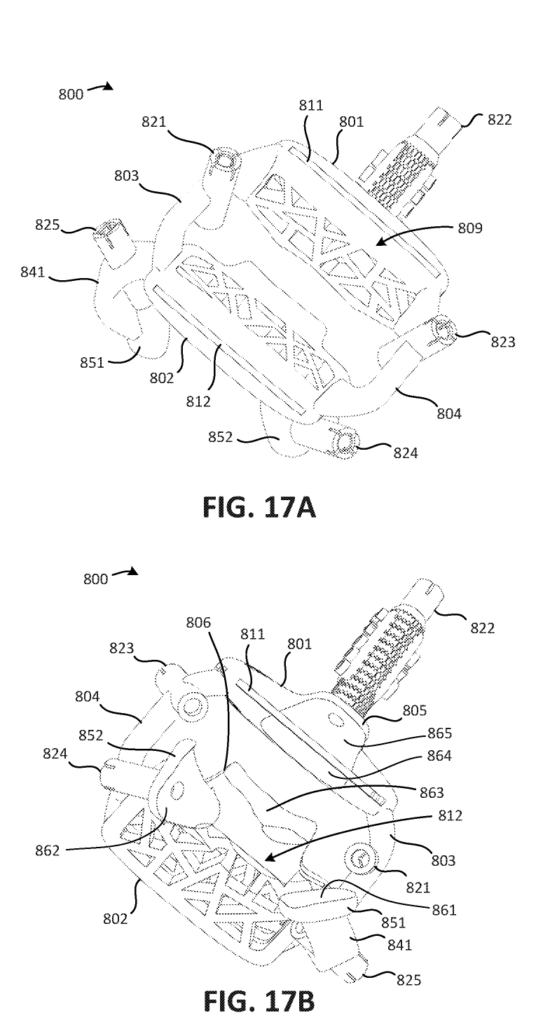

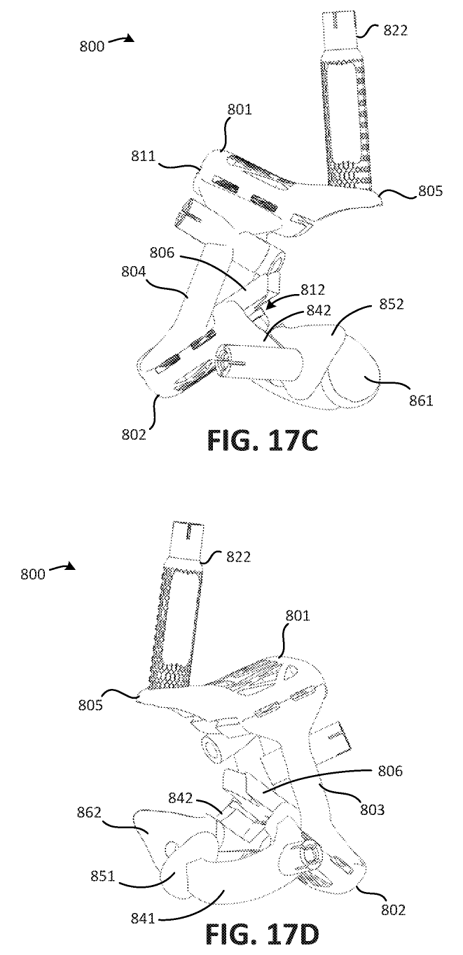

In some embodiments, any portion(s) of the orthopedic guide 100 may include one or more webbed/lattice structures to reduce an amount of material utilized during an additive manufacturing process (e.g., see the lattice structures 809 and 904 shown in FIGS. 17A and 21, as just two non-limiting examples).

In some embodiments, the first guide arm 141 and/or the second guide arm 142 may be selectively broken away from the guide body 101 by the surgeon during the surgical procedure. For example, once the orthopedic guide 100 has been pinned to the bone 10, the first guide arm 141 may be broken off and removed from the guide body 101 to facilitate a subsequent saw blade operation along the cutting guide surface 111 in order to resect a portion of the bone and create the first prepared surface 11 of the bone 10 (e.g., see FIG. 6).

In some embodiments, a first implant adapter 20 may also be coupled to the first prepared surface 11 of the bone 10 (e.g., see FIG. 7). The first implant adapter 20 may be configured to couple with one or more humeral implants by engaging a humeral implant with a first implant adapter rim 21 and/or with one or more first implant adapter features 22 associated with the first implant adapter 20.

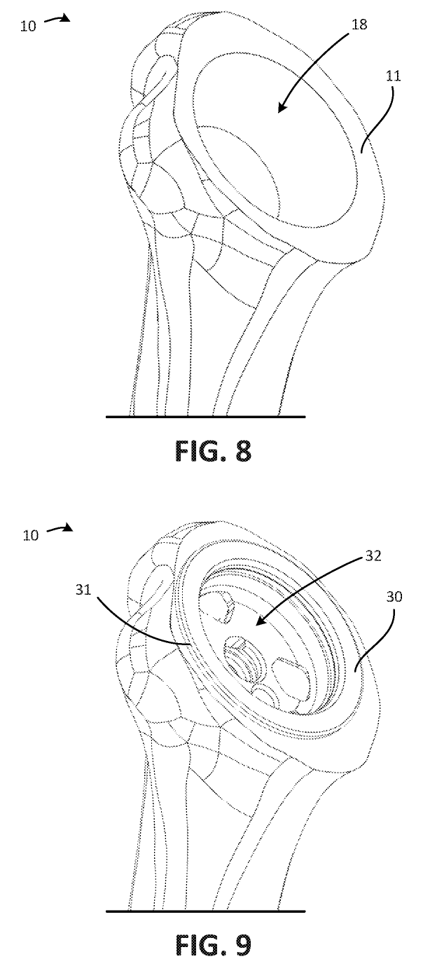

In some embodiments, a bone cavity 18 may also be formed into the humeral head (e.g., see FIG. 8) with a suitable reamer tool (not shown) to receive a second implant adapter 30 therein (e.g., see FIG. 9). The second implant adapter 30 may be configured to couple with one or more humeral implants by engaging a humeral implant with a second implant adapter rim 31 and/or with one or more second implant adapter features 32 disposed within or associated with the second implant adapter 30.

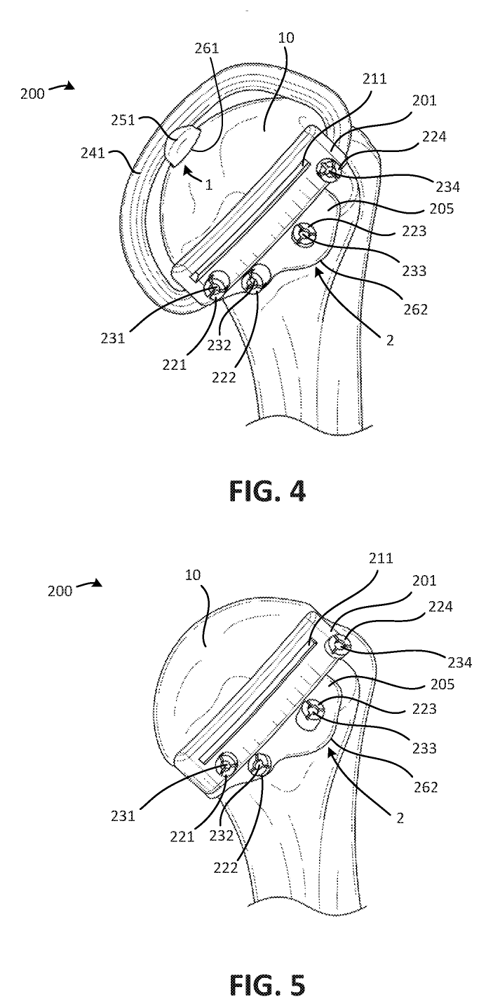

FIGS. 4 and 5 illustrate an orthopedic guide 200 engaged with a bone 10 (e.g., a humeral head, etc.), according to another embodiment of the present disclosure.

In some embodiments, the orthopedic guide 200 may generally include: a guide body 201; a cutting guide slot 211 formed through the guide body 201; a guide body projection 205 that may project away from a bone-facing surface and/or an inferior surface of the guide body 201; a first fixation guide 221 coupled to a proximal surface of the guide body 201 that may include a first fixation guide bore 231 extending through the guide body 201; a second fixation guide 222 coupled to the guide body projection 205 that may include a second fixation guide bore 232 extending therethrough; a third fixation guide 223 coupled to the guide body projection 205 that may include a third fixation guide bore 233 extending therethrough; a fourth fixation guide 224 coupled to a proximal surface of the guide body 201 that may include a fourth fixation guide bore 234 extending through the guide body 201; a first guide arm 241 extending from a first side surface of the guide body 201 to a second side surface of the guide body 201 along a first curved path; a first guide arm bud 251 coupled to the first guide arm 241; a first patient-specific surface 261 formed on the first guide arm bud 251; a second patient-specific surface 262 formed on the bone-facing surface of the guide body 201 and/or on the guide body projection 205 (if present).

However, it will be understood that in other embodiments the orthopedic guide 200 may include any number of guide bodies, cutting guide surfaces/slots, guide body projections, fixation guides/bores, reference guide cannulas, guide arms, guide arm buds, patient-specific surfaces, etc., and/or may incorporate any other feature, morphology, shape, or orientation that is disclosed or contemplated herein with respect to any other guide.

As previously described, FIG. 4 shows how each of the patient-specific surfaces (261, 262) formed on the orthopedic guide 200 can be shaped to correspond to specific anatomical features or areas on the bone of the patient to help position the orthopedic guide 200 at a predetermined/desired location or orientation relative to the bone 10, as each of these patient-specific surfaces (261, 262) engage with their corresponding anatomical feature on the bone 10. As one non-limiting example, FIG. 4 shows how the first patient-specific surface 261 can engage against the first anatomical feature 1 on the bone 10, and the second patient-specific surface 262 can engage against the second anatomical feature 2 on the bone 10. This will prompt the orthopedic guide 200 to “self-locate” into its proper orientation relative to the bone 10 and couple the guide body 201 to the bone 10 with at least one guide feature associated with the guide body 201 positioned at a desired orientation relative to the bone 10, as the patient-specific surfaces (261, 262) engage their corresponding anatomical features (1, 2).

In some embodiments, at least one guide feature associated with the guide body 201 may comprise a reference guide cannula (e.g., see the reference guide cannula 922 shown in FIG. 21, as one non-limiting example), which may be configured to guide an elongate surgical instrument therethrough (e.g., a guide wire, K-wire, drill bit, reference pin, etc.) and into the bone 10 along a desired axis (e.g., a patient-specific axis that may be determined preoperatively, etc.).

In some embodiments, at least one guide feature associated with the guide body 201 may include the cutting guide slot 211 formed through the guide body 201, which may be configured to guide a surgical instrument (e.g., a saw blade, etc.) into the bone 10 along a desired cutting plane trajectory to resect a portion of the bone 10 and create a prepared surface on the bone (e.g., see the first prepared surface 11 of the bone 10 in FIG. 6).

In some embodiments, the orthopedic guide 200 may also include one or more markings that may be printed or otherwise formed on the guide including, but not limited to: one or more orientation features configured to indicate an orientation of the orthopedic guide 200 relative to the bone 10 of the patient, any instructions for use during the surgical procedure, any patient identifier information, any other labeling, etc.

In some embodiments, any portion(s) of the orthopedic guide 200 may include one or more webbed/lattice structures to reduce an amount of material utilized during an additive manufacturing process.

In some embodiments, the first guide arm 241 may be selectively broken away from the guide body 201 by the surgeon during the surgical procedure. For example, once the orthopedic guide 200 has been secured to the bone 10, the first guide arm 241 may be broken off and removed from the guide body 201 (e.g., see FIG. 5) to facilitate a subsequent saw blade operation through the cutting guide slot 211 to resect a portion of the bone and create the first prepared surface 11 on the bone 10 (e.g., see FIG. 6).

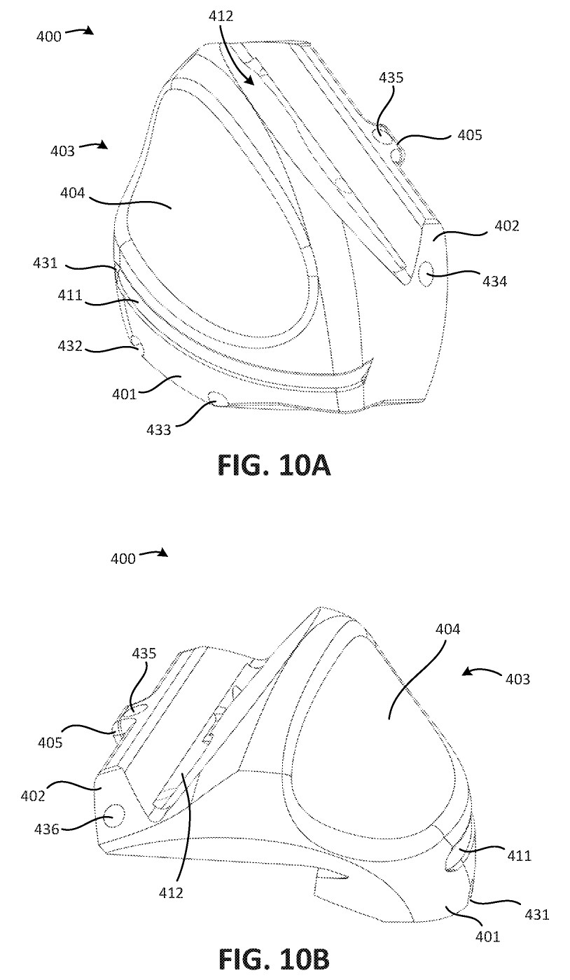

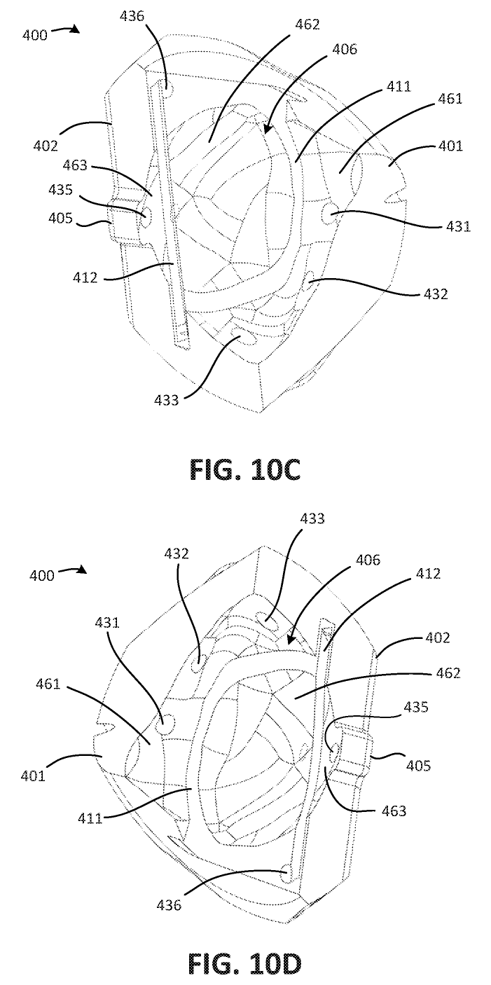

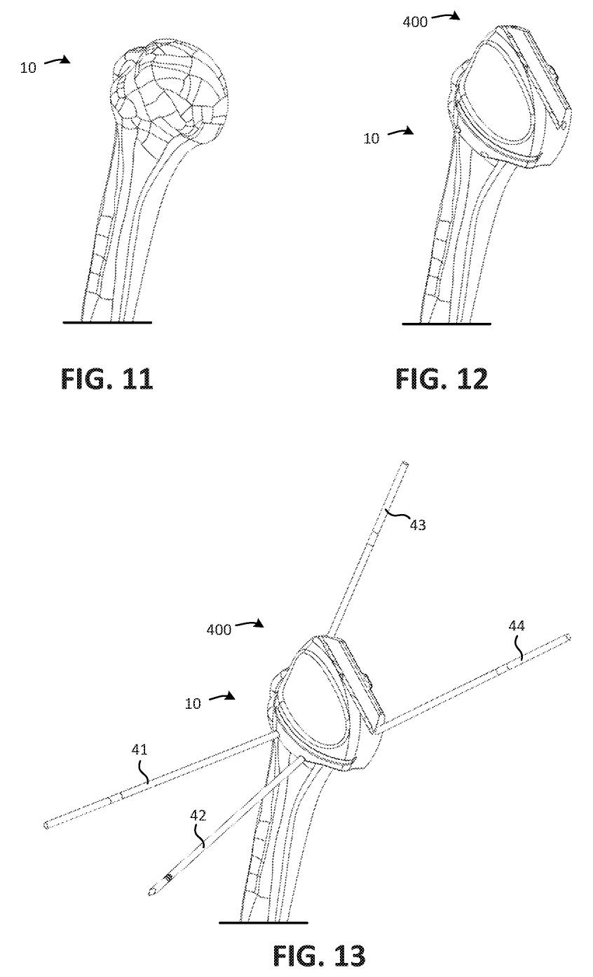

FIGS. 10A-13 illustrate an orthopedic guide 400, according to another embodiment of the present disclosure. Specifically, FIGS. 10A-10D show various perspective views of the orthopedic guide 400, FIG. 11 shows a bone 10 (e.g., a humeral bone, etc.), FIG. 12 shows the orthopedic guide 400 engaged with the bone 10, and FIG. 13 shows the orthopedic guide 400 pinned to the bone 10 via a first securement pin 41, a second securement pin 42, a third securement pin 43, and a fourth securement pin 44, which may be inserted through the orthopedic guide 400 and into the bone 10.

In some embodiments, the orthopedic guide 400 may generally include: a first guide body 401; a second guide body 402; a guide body bridge 403 coupled intermediate the first guide body 401 and the second guide body 402; a guide body bridge recess 404 formed in the guide body bridge 403; a guide body projection 405 protruding from the second guide body 402; a guide body cavity 406 formed in the orthopedic guide 400; a first cutting guide slot 411 formed in the first guide body 401; a second cutting guide slot 412 formed in the second guide body 402; a first fixation guide bore 431, a second fixation guide bore 432, a third fixation guide bore 433, a fourth fixation guide bore 434, a fifth fixation guide bore 435, and a sixth fixation guide bore 436 formed through the orthopedic guide 400; a first patient-specific surface 461, a second patient-specific surface 462, and a third patient-specific surface 463 formed on the orthopedic guide 400 within the guide body cavity 406.

However, it will be understood that in other embodiments the orthopedic guide 400 may include any number of guide bodies, cutting guide slots/surfaces, guide body projections, fixation guides/bores, reference guide cannulas, guide arms, guide arm buds, patient-specific surfaces, etc., and/or may incorporate any other feature, morphology, shape, or orientation that is disclosed or contemplated herein with respect to any other guide.

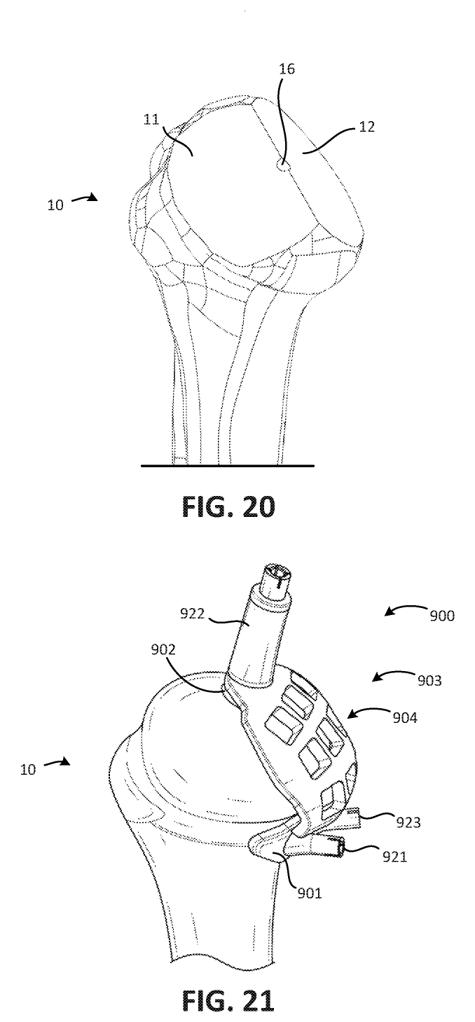

In some embodiments, at least one guide feature associated with the orthopedic guide 400 may include the first cutting guide slot 411 and/or the second cutting guide slot 412, which may be configured to guide a surgical instrument (e.g., a saw blade, etc.) into the bone 10 along one or more desired cutting plane trajectories to resect one or more portions of the bone 10 and create one or more prepared surfaces on the bone 10 (e.g., see the first prepared surface 11 and the second prepared surface 12 on the bone 10 in FIG. 20, which may be angled relative to each other).

In some embodiments, at least one guide feature associated with the orthopedic guide 400 may comprise a reference guide cannula (e.g., see the reference guide cannula 922 shown in FIG. 21, as one non-limiting example), which may be configured to guide an elongate surgical instrument therethrough (e.g., a guide wire, K-wire, drill bit, reference pin, etc.) and into the bone 10 along a desired axis (e.g., a patient-specific axis that may be determined preoperatively, etc.). FIG. 20 shows a reference bone tunnel 16 that may be formed in the bone 10 via an elongate surgical instrument placed through a reference guide cannula, as just one non-limiting example.

In some embodiments, the orthopedic guide 400 may also include one or more markings that may be printed or otherwise formed on the orthopedic guide 400 including, but not limited to: one or more orientation features configured to indicate an orientation of the orthopedic guide 400 relative to the bone 10 of the patient, any instructions for use during the surgical procedure, any patient identifier information, any other labeling, etc.

In some embodiments, any portion(s) of the orthopedic guide 400 may include one or more webbed/lattice structures to reduce an amount of material utilized during an additive manufacturing process.

In some embodiments, the orthopedic guide 400 may also include one or more guide arms extending from the orthopedic guide 400 and configured to engage one or more bony landmarks. The one or more guide arms may also be configured to selectively break away from the orthopedic guide 400 during the surgical procedure, as previously discussed.

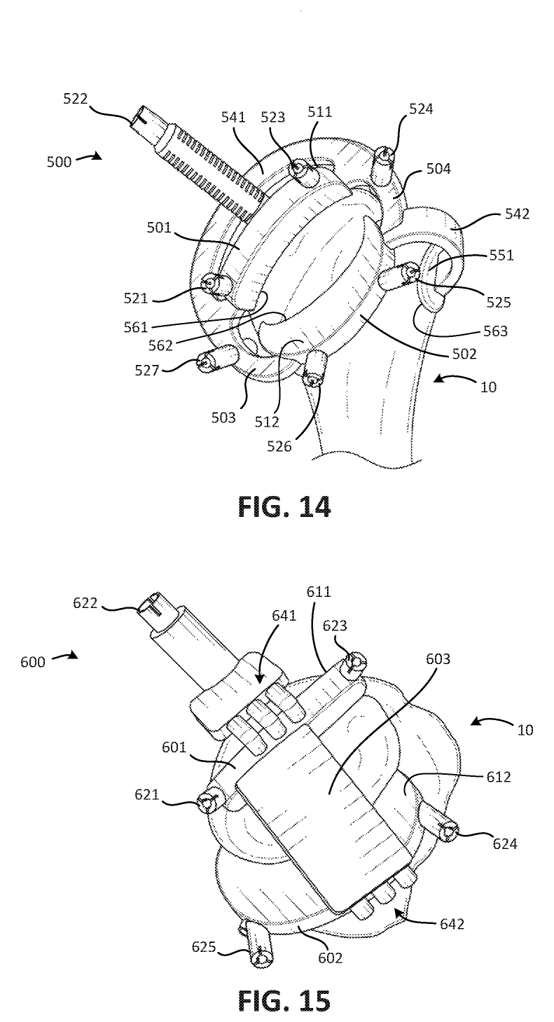

FIG. 14 illustrates an orthopedic guide 500 engaged with a bone 10 (e.g., a humeral head, etc.), according to another embodiment of the present disclosure.

In some embodiments, the orthopedic guide 500 may generally include: a first guide body 501; a second guide body 502; a first guide body bridge 503 and a second guide body bridge 504 coupled intermediate the first guide body 501 and the second guide body 502; a first cutting guide surface 511 on the first guide body 501 and a second cutting guide surface 512 on the second guide body 502; a first fixation guide 521, a second fixation guide 522 (or reference guide cannula), a third fixation guide 523, a fourth fixation guide 524, a fifth fixation guide 525, a sixth fixation guide 526, and a seventh fixation guide 527; a first guide arm 541 extending from a first side surface of the first guide body 501 to a second side surface of the first guide body 501 along a first curved path; a second guide arm 542 extending from an inferior surface of the second guide body 502 distally along a second curved path; a first guide arm bud 551 coupled to the second guide arm 542; a first patient-specific surface 561 formed on a bone-facing surface of the first guide body 501; a second patient-specific surface 562 formed on a bone-facing surface of the second guide body 502; and a third patient-specific surface 563 formed on the first guide arm bud 551.

However, it will be understood that in other embodiments the orthopedic guide 500 may include any number of guide bodies, cutting guide surfaces/slots, guide body projections, fixation guides/bores, reference guide cannulas, guide arms, guide arm buds, patient-specific surfaces, etc., and/or may incorporate any other feature, morphology, shape, or orientation that is disclosed or contemplated herein with respect to any other guide.

In some embodiments, at least one guide feature associated with the orthopedic guide 500 may include the first cutting guide surface 511 and/or the second cutting guide surface 512, which may be configured to guide a surgical instrument (e.g., a saw blade, etc.) into the bone 10 along one or more desired cutting plane trajectories to resect one or more portions of the bone 10 and create one or more prepared surfaces on the bone 10 (e.g., see the first prepared surface 11 and the second prepared surface 12 on the bone 10 shown in FIG. 20, which may be angled relative to each other).

In some embodiments, at least one guide feature associated with the orthopedic guide 500 may include the second fixation guide 522 or reference guide cannula, which may be configured to guide an elongate surgical instrument (e.g., a guide wire, K-wire, drill bit, reference pin, etc.) into the bone 10 along a desired patient-specific axis. FIG. 20 shows a reference bone tunnel 16 that may be formed in the bone 10 via an elongate surgical instrument placed through a reference guide cannula and into the bone 10.

In some embodiments, the orthopedic guide 500 may also include one or more markings that may be printed or otherwise formed on the orthopedic guide 500 including, but not limited to: one or more orientation features configured to indicate an orientation of the orthopedic guide 500 relative to the bone 10 of the patient, any instructions for use during the surgical procedure, any patient identifier information, any other labeling, etc.

In some embodiments, any portion(s) of the orthopedic guide 500 may include one or more webbed/lattice structures to reduce an amount of material utilized during an additive manufacturing process.

In some embodiments, any portion(s) of the orthopedic guide 500 may be configured to selectively break away from the orthopedic guide 500 during the surgical procedure, as previously described herein.

FIG. 15 illustrates an orthopedic guide 600 engaged with a bone 10 (e.g., a humeral head, etc.), according to another embodiment of the present disclosure.

In some embodiments, the orthopedic guide 600 may generally include: a first guide body 601; a second guide body 602; a guide body bridge 603 coupled intermediate the first guide body 601 and the second guide body 602; a first cutting guide surface 611 on the first guide body 601 and a second cutting guide surface 612 on the second guide body 602; a first fixation guide 621, a second fixation guide 622 (or reference guide cannula), a third fixation guide 623, a fourth fixation guide 624, and a fifth fixation guide 625; at least one first coupling arm 641 extending between the second fixation guide 622 and the first guide body 601 and/or the guide body bridge 603; and at least one second coupling arm 642 extending between the second guide body 602 and the guide body bridge 603.

However, it will be understood that in other embodiments the orthopedic guide 600 may include any number of guide bodies, cutting guide surfaces/slots, guide body projections, fixation guides/bores, reference guide cannulas, guide arms, guide arm buds, coupling arms, patient-specific surfaces, etc., and/or may incorporate any other feature, morphology, shape, or orientation that is disclosed or contemplated herein with respect to any other guide.

In some embodiments, any portion(s) of the orthopedic guide 600 may be configured to be selectively broken away from the orthopedic guide 600 by the surgeon during the surgical procedure, as previously described herein. For example, the coupling arms may be easily broken to remove the guide body bridge 603 and/or the second fixation guide 622 from the orthopedic guide 600.

In some embodiments, at least one guide feature associated with the orthopedic guide 600 may include the first cutting guide surface 611 and/or the second cutting guide surface 612, which may be configured to guide a surgical instrument (e.g., a saw blade, etc.) into the bone 10 along one or more desired cutting plane trajectories to resect one or more portions of the bone 10 and create one or more prepared surfaces on the bone 10 (e.g., see the first prepared surface 11 and the second prepared surface 12 on the bone 10 shown in FIG. 20, which may be angled relative to each other).

In some embodiments, at least one guide feature associated with the orthopedic guide 600 may include the second fixation guide 622 or reference guide cannula, which may be configured to guide an elongate surgical instrument (e.g., a guide wire, K-wire, drill bit, reference pin, etc.) into the bone 10 along a desired patient-specific axis. FIG. 20 shows a reference bone tunnel 16 that may be formed in the bone 10 via an elongate surgical instrument that may be placed through the reference guide cannula and into the bone 10.

In some embodiments, the orthopedic guide 600 may also include one or more markings that may be printed or otherwise formed on the orthopedic guide 600 including, but not limited to: one or more orientation features configured to indicate an orientation of the orthopedic guide 600 relative to the bone 10 of the patient, any instructions for use during the surgical procedure, any patient identifier information, any other labeling, etc.

In some embodiments, any portion(s) of the orthopedic guide 600 may include one or more webbed/lattice structures to reduce an amount of material utilized during an additive manufacturing process.

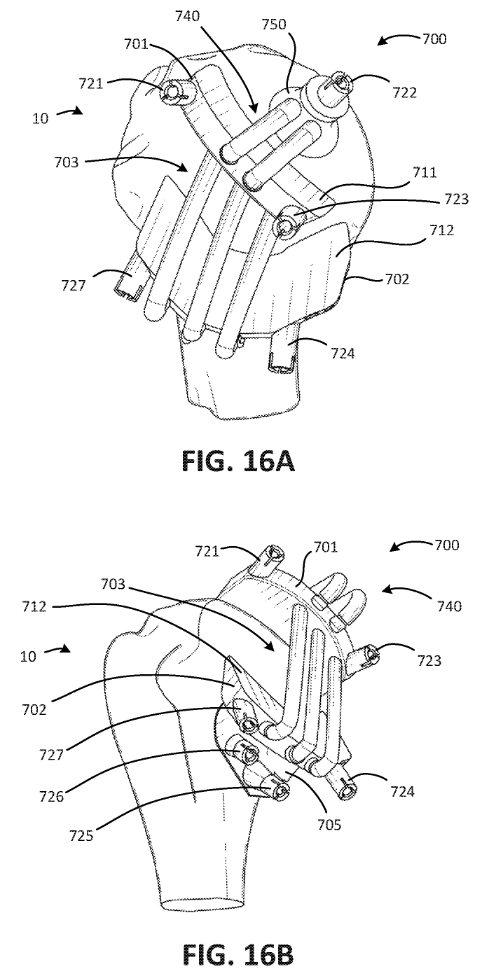

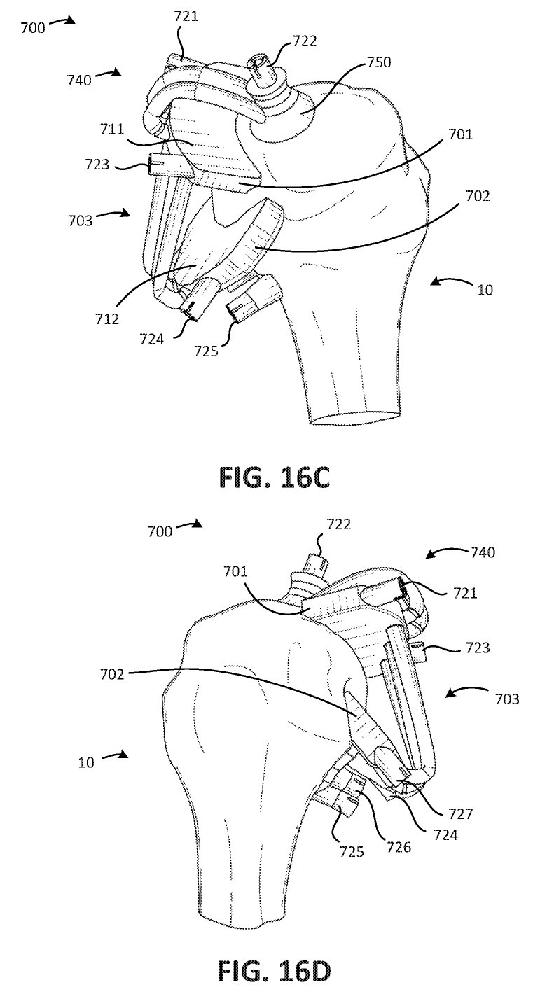

FIGS. 16A-16D illustrate various views of an orthopedic guide 700 engaged with a bone 10 (e.g., a humeral head, etc.), according to another embodiment of the present disclosure.

In some embodiments, the orthopedic guide 700 may generally include: a first guide body 701; a second guide body 702; at least one guide body bridge arm 703 coupled intermediate the first guide body 701 and the second guide body 702; a guide body projection 705 extending from the second guide body 702; a first cutting guide surface 711 on the first guide body 701 and a second cutting guide surface 712 on the second guide body 702; a first fixation guide 721, a second fixation guide 722 (or reference guide cannula), a third fixation guide 723, a fourth fixation guide 724, a fifth fixation guide 725, sixth fixation guide 726, and a seventh fixation guide 727; at least one guide arm bud or coupling arm bud 750; and at least one guide arm or coupling arm 740 extending between the at least one guide arm bud or coupling arm bud 750 and the first guide body 701.

However, it will be understood that in other embodiments the orthopedic guide 700 may include any number of guide bodies, cutting guide surfaces/slots, guide body projections, fixation guides/bores, reference guide cannulas, guide arms, guide arm buds, coupling arms, patient-specific surfaces, etc., and/or may incorporate any other feature, orientation, shape, or morphology that is disclosed or contemplated herein with respect to any other guide.

In some embodiments, any portion(s) of the orthopedic guide 700 may be configured to be selectively broken away from the orthopedic guide 700 by the surgeon during the surgical procedure. For example, the coupling/bridge arms may be easily broken and removed from the orthopedic guide 700.

In some embodiments, at least one guide feature associated with the orthopedic guide 700 may include the first cutting guide surface 711 and/or the second cutting guide surface 712, which may be configured to guide a surgical instrument (e.g., a saw blade, etc.) into the bone 10 along one or more desired cutting plane trajectories to resect one or more portions of the bone 10 and create one or more prepared surfaces on the bone 10 (e.g., see FIG. 20).

In some embodiments, at least one guide feature associated with the orthopedic guide 700 may include the second fixation guide 722 or reference guide cannula, which may be configured to guide an elongate surgical instrument (e.g., a guide wire, K-wire, drill bit, reference pin, etc.) into the bone 10 along a desired patient-specific axis. FIG. 20 shows a reference bone tunnel 16 that may be formed in the bone 10 via an elongate surgical instrument that may be placed through the reference guide cannula and into the bone 10.

In some embodiments, the orthopedic guide 700 may also include one or more markings that may be printed or otherwise formed on the orthopedic guide 700 including, but not limited to: one or more orientation features configured to indicate an orientation of the orthopedic guide 700 relative to the bone 10 of the patient, any instructions for use during the surgical procedure, any patient identifier information, any other labeling, etc.

In some embodiments, any portion(s) of the orthopedic guide 700 may include one or more webbed/lattice structures to reduce an amount of material utilized during an additive manufacturing process.

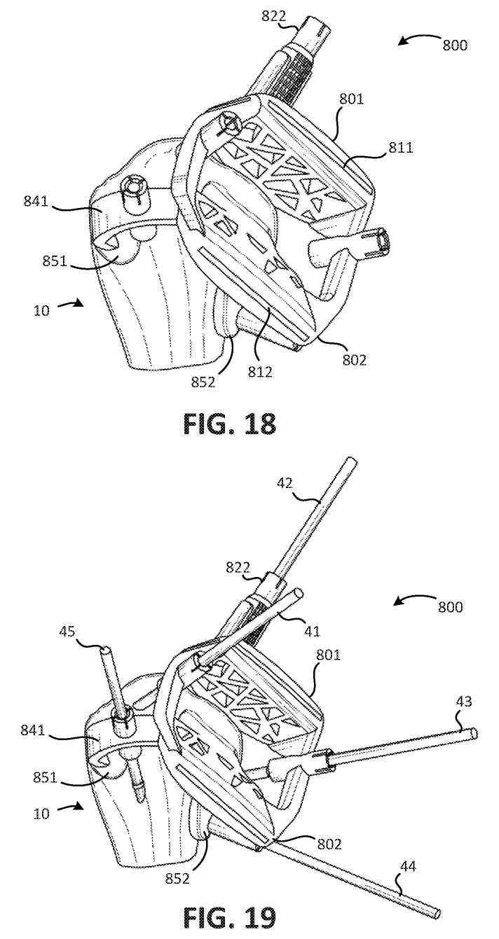

FIGS. 17A-19 illustrate various views of an orthopedic guide 800, according to another embodiment of the present disclosure, which may be coupled to a bone 10 (e.g., a humeral head, etc.) via the first securement pin 41, the second securement pin 42 (or reference pin), the third securement pin 43, the fourth securement pin 44, and/or a fifth securement pin 45, as shown in FIG. 19.

In some embodiments, the orthopedic guide 800 may generally include: a first guide body 801; a second guide body 802; a first guide body bridge 803 and/or a second guide body bridge 804 that may be intermediately coupled between the first guide body 801 and/or the second guide body 802; a first guide body projection 805 extending from the first guide body 801; a second guide body projection 806 extending from the second guide body 802; a first cutting guide slot 811 formed in the first guide body 801; a second cutting guide slot 812 formed in the second guide body 802; a first fixation guide 821, a second fixation guide 822 (or reference guide cannula), a third fixation guide 823, a fourth fixation guide 824, and a fifth fixation guide 825; a first guide arm 841 projecting distally from the second guide body 802 along a first curved path; a second guide arm 842 projecting distally from the second guide body 802; a first guide arm bud 851 coupled to the first guide arm 841; a second guide arm bud 852 coupled to the second guide arm 842; a first patient-specific surface 861 formed on the first guide arm bud 851; a second patient-specific surface 862 formed on the second guide arm bud 852; a third patient-specific surface 863 formed on the second guide body projection 806; a fourth patient-specific surface 864 formed on the first guide body 801; and a fifth patient-specific surface 865 formed on the first guide body projection 805.

However, it will be understood that in other embodiments the orthopedic guide 800 may include any number of guide bodies, cutting guide surfaces/slots, guide body projections, fixation guides/bores, reference guide cannulas, guide arms, guide arm buds, coupling arms, patient-specific surfaces, etc., and/or may incorporate any other feature, morphology, shape or orientation that is disclosed or contemplated herein with respect to any other guide.

In some embodiments, at least one guide feature associated with the orthopedic guide 800 may include the first cutting guide slot 811 and/or the second cutting guide slot 812, which may be configured to guide a surgical instrument (e.g., a saw blade, etc.) into the bone 10 along one or more desired cutting plane trajectories to resect one or more portions of the bone 10 and create one or more prepared surfaces on the bone 10 (e.g., see FIG. 20).

In some embodiments, at least one guide feature associated with the orthopedic guide 800 may include the second fixation guide 822 or reference guide cannula, which may be configured to guide an elongate surgical instrument (e.g., a guide wire, K-wire, drill bit, reference pin, etc.) into the bone 10 along a desired patient-specific axis. FIG. 20 shows a reference bone tunnel 16 that may be formed in the bone 10 via an elongate surgical instrument placed through the reference guide cannula and into the bone 10.

In some embodiments, the orthopedic guide 800 may also include one or more markings that may be printed or otherwise formed on the orthopedic guide 800 including, but not limited to: one or more orientation features configured to indicate an orientation of the orthopedic guide 800 relative to the bone 10 of the patient, any instructions for use during the surgical procedure, any patient identifier information, any other labeling, etc.

In some embodiments, any portion(s) of the orthopedic guide 800 may include one or more webbed/lattice structures or a lattice structure 809 (e.g., see the lattice structures formed in the first guide body 801 and the second guide body 802 in FIG. 17A) to reduce an amount of material utilized during an additive manufacturing process and reduce cost.

In some embodiments, any portion(s) of the orthopedic guide 800 may be configured to selectively break away from the orthopedic guide 800 during the surgical procedure, as previously described herein.

FIG. 21 illustrates an orthopedic guide 900 engaged with a bone 10 (e.g., a humeral head, etc.), according to another embodiment of the present disclosure.

In some embodiments, the orthopedic guide 900 may generally include: a first guide body 901; a second guide body 902; a guide body bridge 903 coupled intermediate the first guide body 901 and the second guide body 902 comprising a lattice structure 904; a first fixation guide 921, a second fixation guide 922 (or reference guide cannula), and a third fixation guide 923.

However, it will be understood that in other embodiments the orthopedic guide 900 may include any number of guide bodies, cutting guide surfaces/slots, guide body projections, fixation guides/bores, reference guide cannulas, guide arms, guide arm buds, coupling arms, patient-specific surfaces, etc., and/or may incorporate any other feature, morphology, shape, or orientation that is disclosed or contemplated herein with respect to any other guide.

In some embodiments, any portion(s) of the orthopedic guide 900 may be configured to be selectively broken away and removed from the orthopedic guide 900 by the surgeon during the surgical procedure, as previously described herein. For example, the guide body bridge 903 may be broken off and removed from the orthopedic guide 900, etc.

In some embodiments, at least one guide feature associated with the orthopedic guide 900 may include the second fixation guide 922 or reference guide cannula, which may be configured to guide an elongate surgical instrument (e.g., a guide wire, K-wire, drill bit, reference pin, etc.) into the bone 10 along a desired patient-specific axis.

In some embodiments, the orthopedic guide 900 may also include one or more markings that may be printed or otherwise formed on the orthopedic guide 900 including, but not limited to: one or more orientation features configured to indicate an orientation of the orthopedic guide 900 relative to the bone 10 of the patient, any instructions for use during the surgical procedure, any patient identifier information, any other labeling, etc.

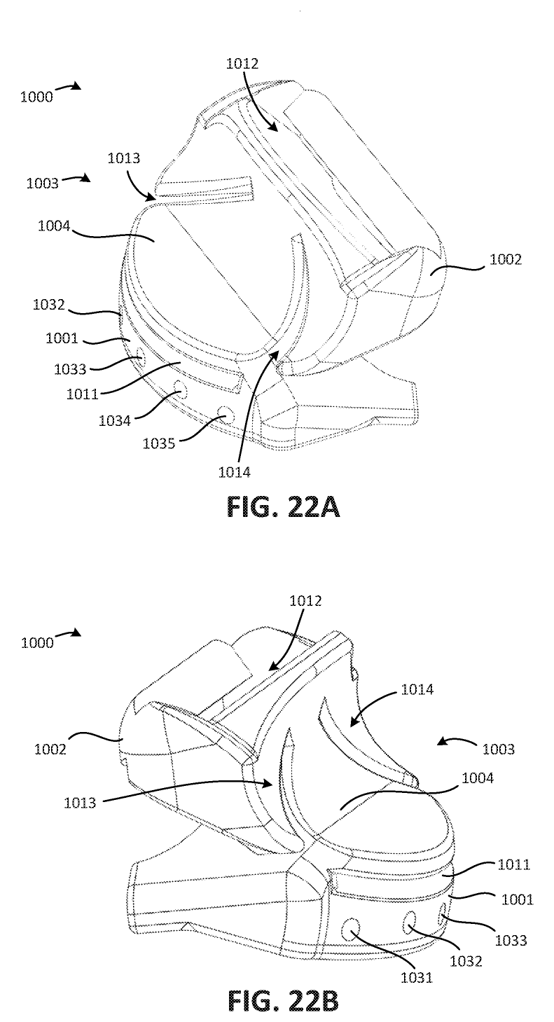

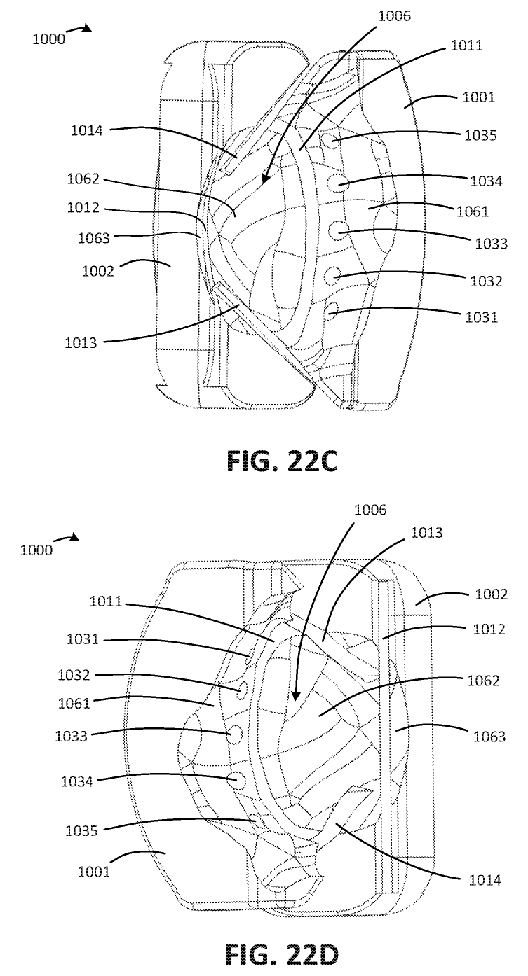

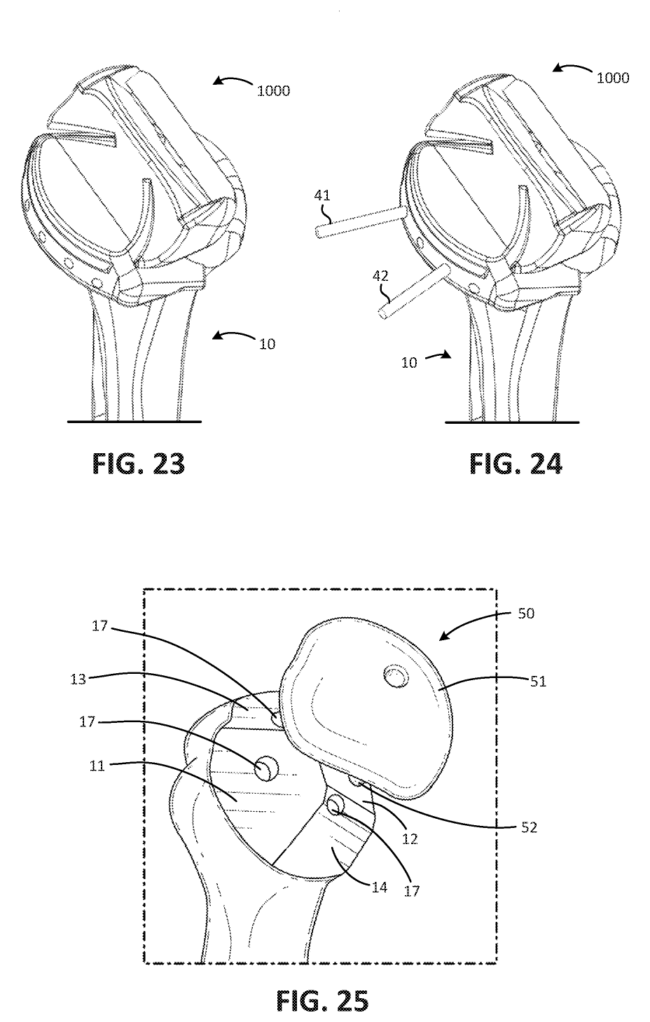

FIGS. 22A-24 illustrate an orthopedic guide 1000, according to another embodiment of the present disclosure. Specifically, FIGS. 22A-22D show various perspective views of the orthopedic guide 1000, FIG. 23 shows the orthopedic guide 1000 engaged with the bone 10 (e.g., a humeral head, etc.), and FIG. 24 shows the orthopedic guide 1000 pinned to the bone 10 via a first securement pin 41 and a second securement pin 42, which may be inserted through the orthopedic guide 1000 and into the bone 10.

In some embodiments, the orthopedic guide 1000 may generally include: a first guide body 1001; a second guide body 1002; a guide body bridge 1003 coupled intermediate the first guide body 1001 and the second guide body 1002; a guide body bridge recess 1004 formed in the guide body bridge 1003; a guide body cavity 1006 formed in the orthopedic guide 1000; a first cutting guide slot 1011 formed in the first guide body 1001; a second cutting guide slot 1012 formed in the second guide body 1002; a third cutting guide slot 1013 formed in the guide body bridge 1003; a fourth cutting guide slot 1014 formed in the guide body bridge 1003; a first fixation guide bore 1031, a second fixation guide bore 1032, a third fixation guide bore 1033, a fourth fixation guide bore 1034, and a fifth fixation guide bore 1035 formed through the orthopedic guide 1000; a first patient-specific surface 1061, a second patient-specific surface 1062, and a third patient-specific surface 1063 formed on the orthopedic guide 1000 within the guide body cavity 1006.

However, it will be understood that in other embodiments the orthopedic guide 1000 may include any number of guide bodies, cutting guide slots/surfaces, guide body projections, fixation guides/bores, reference guide cannulas, guide arms, guide arm buds, patient-specific surfaces, etc., and/or may incorporate any other feature, morphology, shape, or orientation that is disclosed or contemplated herein with respect to any other guide.

In some embodiments, at least one guide feature associated with the orthopedic guide 1000 may include the first cutting guide slot 1011, the second cutting guide slot 1012, the third cutting guide slot 1013, and/or the fourth cutting guide slot 1014 which may be configured to guide a surgical instrument (e.g., a saw blade, etc.) into the bone 10 along one or more desired cutting plane trajectories to resect one or more portions of the bone 10 and create one or more prepared surfaces on the bone 10 (e.g., see the first prepared surface 11, the second prepared surface 12, the third prepared surface 13, and the fourth prepared surface 14 on the bone 10 in FIG. 25, which may be angled relative to each other).

In some embodiments, at least one guide feature associated with the orthopedic guide 1000 may comprise a reference guide cannula (e.g., see the reference guide cannula 922 shown in FIG. 21, as one non-limiting example), which may be configured to guide an elongate surgical instrument therethrough (e.g., a guide wire, K-wire, drill bit, reference pin, etc.) and into the bone 10 along a desired axis (e.g., a patient-specific axis that may be determined preoperatively, etc.). FIG. 20 shows a reference bone tunnel 16 that may be formed in the bone 10 via an elongate surgical instrument placed through a reference guide cannula, as one non-limiting example.

In some embodiments, the orthopedic guide 1000 may also include one or more markings that may be printed or otherwise formed on the orthopedic guide 1000 including, but not limited to: one or more orientation features configured to indicate an orientation of the orthopedic guide 1000 relative to the bone 10 of the patient, any instructions for use during the surgical procedure, any patient identifier information, any other labeling, etc.

In some embodiments, any portion(s) of the orthopedic guide 1000 may include one or more webbed/lattice structures to reduce an amount of material utilized during an additive manufacturing process, as previously described.

In some embodiments, the orthopedic guide 1000 may also include one or more guide arms extending from the orthopedic guide 1000 and configured to engage one or more bony landmarks. The one or more guide arms may also be configured to selectively break away from the orthopedic guide 1000 during the surgical procedure.

FIG. 25 shows the four prepared bone surfaces with one or more implant bone holes 17 formed therein and configured to receive one or more humeral implant dowels 52 of a humeral implant 50 therein. The humeral implant 50 shown in this example includes a humeral implant articular surface 51 that may be convex in shape (e.g., for an anatomical procedure). However, other humeral implant embodiments may include a concave articular surface (e.g., for a reverse procedure).

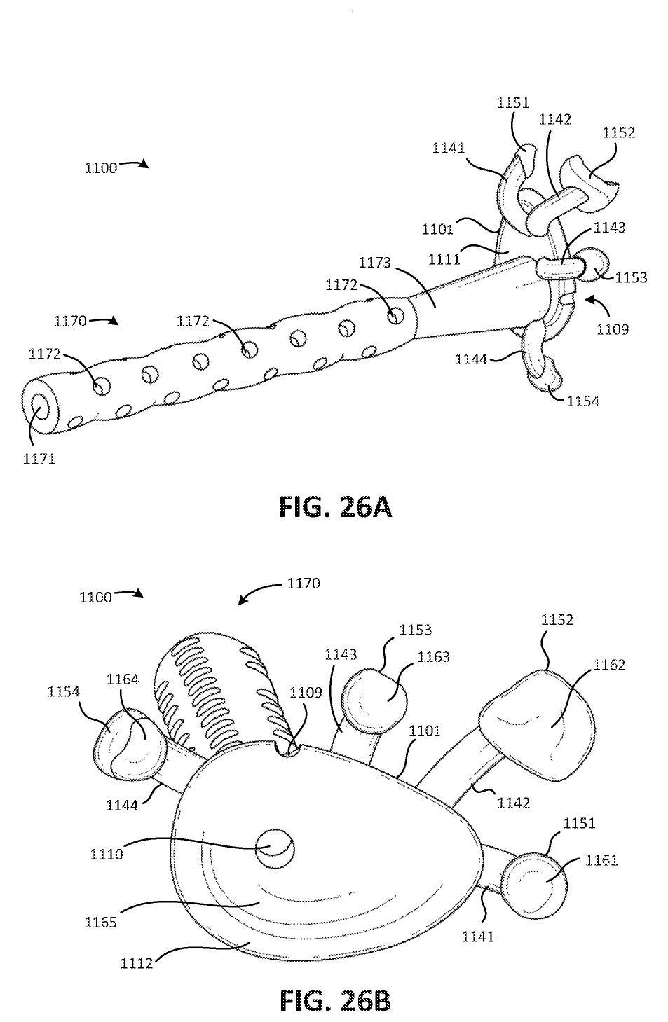

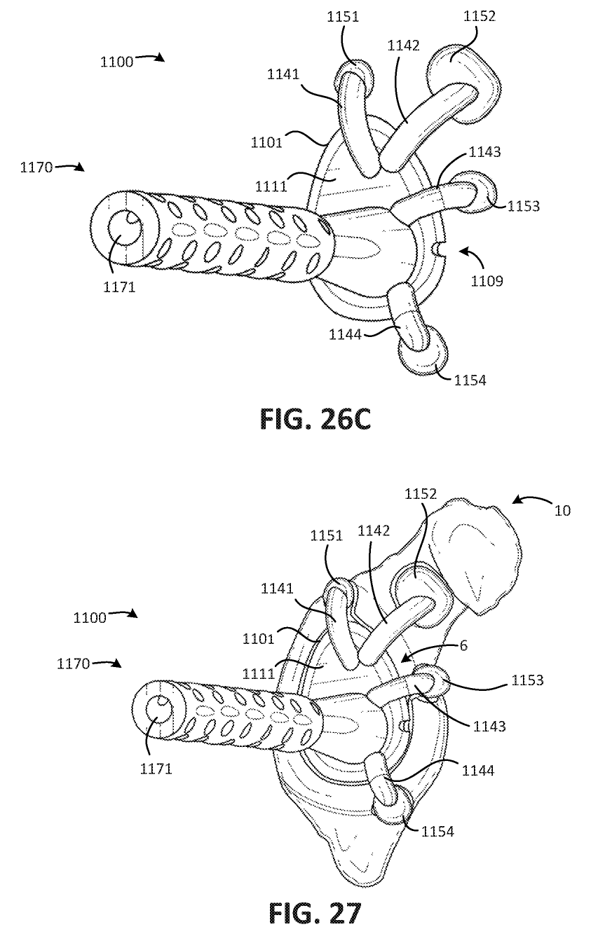

FIGS. 26A-27 illustrate various perspective views of an orthopedic guide 1100 that can engage with a bone 10 (e.g., a glenoid cavity 6 on a scapula bone, as shown in FIG. 27), according to another embodiment of the present disclosure.

In some embodiments, the orthopedic guide 1100 may generally include: a guide body 1101; an orientation feature 1109 or marking formed in or on the guide body 1101; a guide body center punch hole 1110 formed through the guide body 1101; a bone-facing surface 1112 on a first side of the guide body 1101; an obverse surface 1111 on a second side of the guide body 1101, opposite the bone-facing surface 1112; a first guide arm 1141 projecting from the guide body 1101; a second guide arm 1142 projecting from the guide body 1101; a third guide arm 1143 projecting from the guide body 1101; a fourth guide arm 1144 projecting from the guide body 1101; a first guide arm bud 1151 coupled to the first guide arm 1141; a second guide arm bud 1152 coupled to the second guide arm 1142; a third guide arm bud 1153 coupled to the third guide arm 1143; a fourth guide arm bud 1154 coupled to the fourth guide arm 1144; a first patient-specific surface 1161 formed on the first guide arm bud 1151; a second patient-specific surface 1162 formed on the second guide arm bud 1152; a third patient-specific surface 1163 formed on the third guide arm bud 1153; a fourth patient-specific surface 1164 formed on the fourth guide arm bud 1154; a fifth patient-specific surface 1165 formed on bone-facing surface 1112 of the guide body 1101; a guide handle 1170; a guide handle attachment feature 1173 coupling the guide handle 1170 to the guide body 1101; a guide handle longitudinal cannula 1171 formed through the guide handle 1170 and/or the guide handle attachment feature 1173; and one or more transverse guide handle holes 1172 formed through the guide handle 1170.

However, it will be understood that in other embodiments the orthopedic guide 1100 may include any number of guide bodies, guide body projections, fixation guides/bores, guide cannulas/holes, guide arms, guide arm buds, patient-specific surfaces, handle structures, etc., and/or may incorporate any other feature, morphology, shape or orientation that is disclosed or contemplated herein with respect to any other guide.

In some embodiments, at least one guide feature associated with the guide body 1101 may comprise the guide handle longitudinal cannula 1171, which may be configured to guide an elongate surgical instrument (e.g., a guide wire, K-wire, drill bit, reference pin, etc.) into the bone 10 along a desired axis (e.g., a patient-specific axis that may be determined preoperatively, etc.).

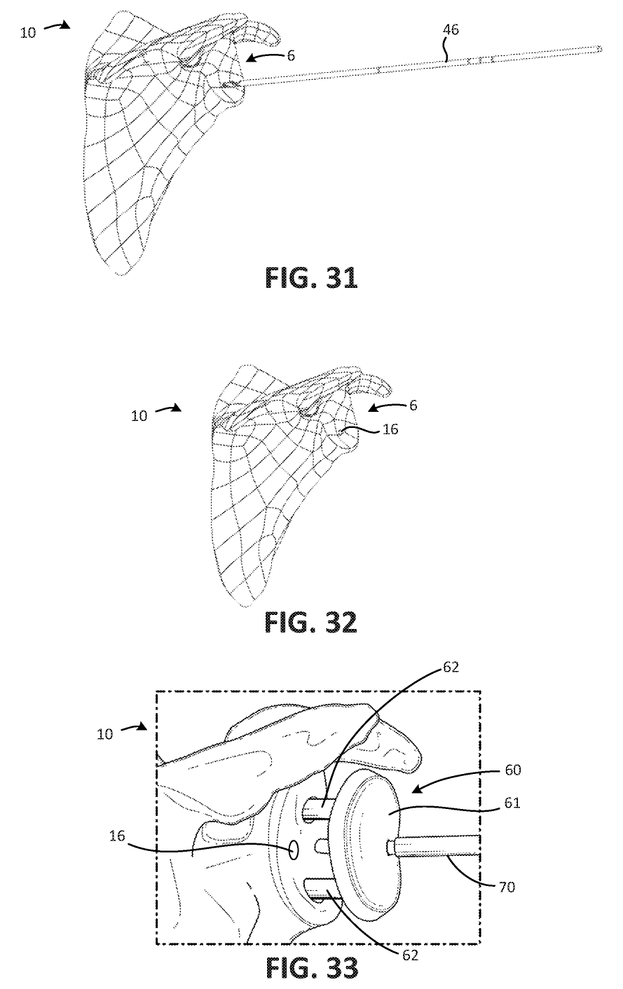

FIG. 31 shows an elongate surgical instrument 46 that may be oriented along a desired patient-specific axis and placed in the bone 10 with the aid of the orthopedic guide 1100.

Once the elongate surgical instrument 46 is inserted into the bone 10 along the desired patient-specific axis, the elongate surgical instrument 46 may then be utilized to guide another surgical instrument (or an implant) relative to the bone 10 to a desired location onto or within the bone. For example, the elongate surgical instrument 46 may be utilized to guide a cannulated reamer (not shown) towards a surface of the glenoid cavity 6 during a reaming operation to ream the glenoid cavity 6 and achieve a desired shape thereon, and/or remove a specific amount of bone from the glenoid cavity 6 during the reaming operation.

FIG. 32 shows a prepared glenoid cavity 6 with the elongate surgical instrument 46 removed from the bone 10, leaving behind a reference bone tunnel 16 formed therein.

FIG. 33 shows a close-up view of the prepared glenoid cavity 6 with one or more bone tunnels formed therein that may be configured to receive one or more glenoid implant dowels 62 of a glenoid implant 60. FIG. 33 shows the glenoid implant 60 being inserted onto the glenoid cavity 6 via an inserter tool 70. In this example, the glenoid implant 60 comprises a glenoid implant articular surface 61 that comprises a concave shape (e.g., for an anatomic procedure). However, it will be understood that other glenoid implants may be utilized that include a convex articular surface (e.g., for a reverse procedure).

In some embodiments, the orthopedic guide 1100 may include one or more markings (in place of, or in addition to the orientation feature 1109), which may be printed or otherwise formed on the orthopedic guide 1100 including, but not limited to: one or more orientation features configured to indicate an orientation of the orthopedic guide 1100 relative to the bone 10 of the patient, any instructions for use during the surgical procedure, any patient identifier information, any other labeling, etc.

In some embodiments, any portion(s) of the orthopedic guide 1100 may include one or more webbed/lattice structures to reduce an amount of material utilized during an additive manufacturing process.

In some embodiments, one or more of the guide arms may be configured to be selectively broken away from the guide body 1101 by the surgeon during the surgical procedure, as previously discussed herein.

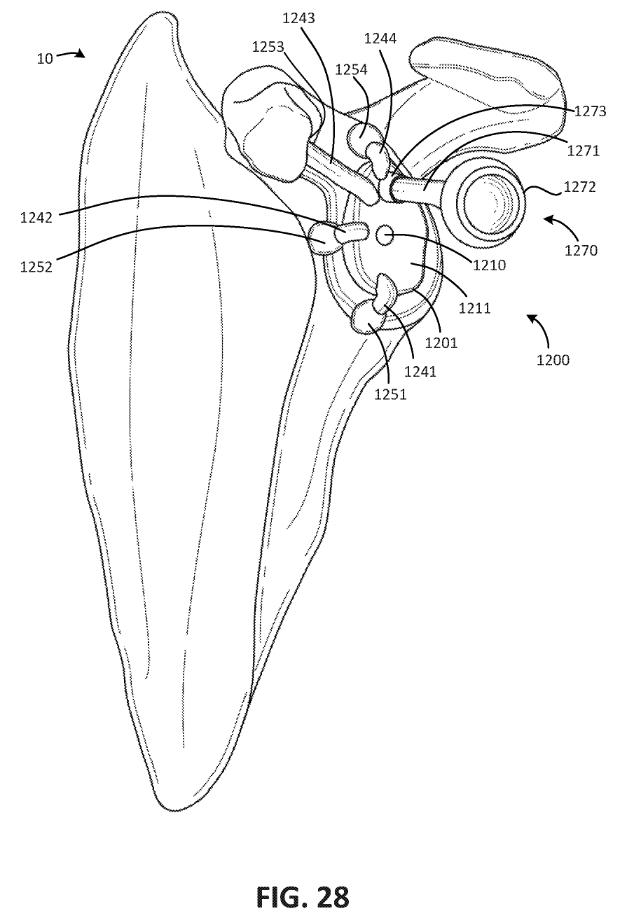

FIG. 28 illustrates an orthopedic guide 1200 engaged with a bone 10 (e.g., a glenoid cavity 6 on a scapula bone), according to another embodiment of the present disclosure.

In some embodiments, the orthopedic guide 1200 may generally include: a guide body 1201; a guide body center punch hole 1210 formed through the guide body 1201; a bone-facing surface on a first side of the guide body 1201; an obverse surface 1211 on a second side of the guide body 1201, opposite the bone-facing surface; a first guide arm 1241 projecting from the guide body 1201; a second guide arm 1242 projecting from the guide body 1201; a third guide arm 1243 projecting from the guide body 1201; a fourth guide arm 1244 projecting from the guide body 1201; a first guide arm bud 1251 coupled to the first guide arm 1241; a second guide arm bud 1252 coupled to the second guide arm 1242; a third guide arm bud 1253 coupled to the third guide arm 1243; a fourth guide arm bud 1254 coupled to the fourth guide arm 1244; one or more patient-specific surfaces, that may be analogous to the orthopedic guide 1100 shown in FIGS. 26C-27; an offset guide handle 1270; an offset guide handle attachment feature 1273 that couples the offset guide handle 1270 to the guide body 1201; an offset guide handle shaft 1271; and an offset guide handle head 1272.

However, it will be understood that in other embodiments the orthopedic guide 1200 may include any number of guide bodies, guide body projections, fixation guides/bores, guide holes/cannulas, guide arms, guide arm buds, patient-specific surfaces, handle structures, etc., and/or may incorporate any other feature, morphology, shape or orientation that is disclosed or contemplated herein with respect to any other guide.

In some embodiments, at least one guide feature associated with the guide body 1201 may comprise the guide body center punch hole 1210, which may be configured to guide an elongate surgical instrument (e.g., a guide wire, K-wire, drill bit, reference pin, etc.) into the bone 10 along a desired axis (e.g., a patient-specific axis that may be determined preoperatively, etc.). Once the elongate surgical instrument is inserted into the bone 10 along the desired patient-specific axis, the elongate surgical instrument may then be utilized to guide another surgical instrument (or an implant) relative to the bone 10 to a desired location onto or within the bone. For example, the elongate surgical instrument may be utilized to guide a cannulated reamer towards a surface of the bone 10 during a reaming operation to shape the glenoid cavity 6, etc.

In some embodiments, the orthopedic guide 1200 may include one or more markings which may be printed or otherwise formed on the orthopedic guide 1200 including, but not limited to: one or more orientation features configured to indicate an orientation of the orthopedic guide 1200 relative to the bone 10 of the patient, any instructions for use during the surgical procedure, any patient identifier information, any other labeling, etc.

In some embodiments, any portion(s) of the orthopedic guide 1200 may include one or more webbed/lattice structures to reduce an amount of material utilized during an additive manufacturing process.

In some embodiments, one or more of the guide arms may be configured to be selectively broken away from the guide body 1201 by the surgeon during the surgical procedure, as previously described herein.

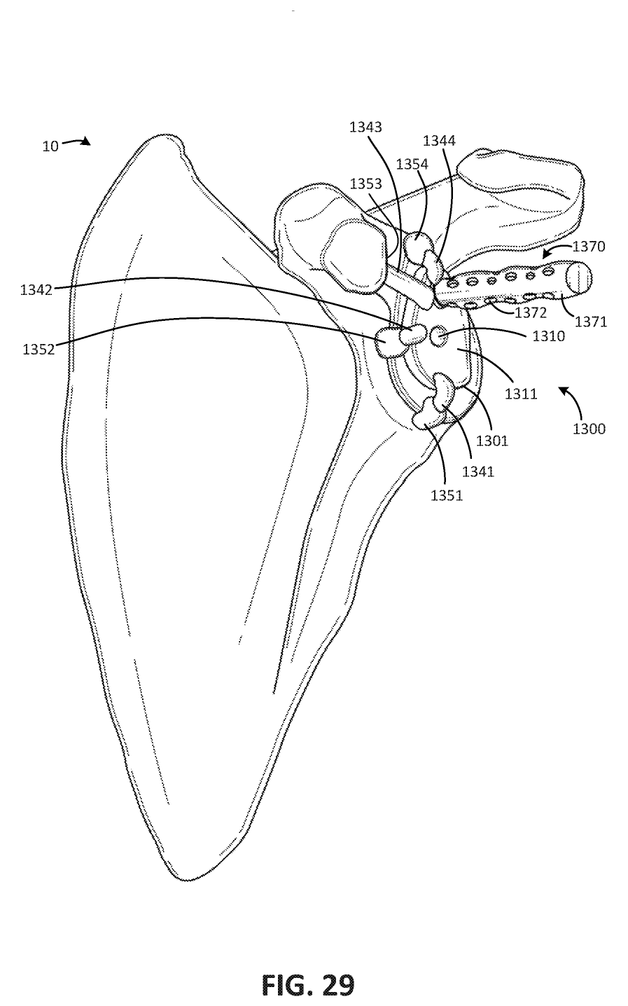

FIG. 29 illustrates an orthopedic guide 1300 engaged with a bone 10 (e.g., a glenoid cavity 6 on a scapula bone), according to another embodiment of the present disclosure.

In some embodiments, the orthopedic guide 1300 may generally include: a guide body 1301; a guide body center punch hole 1310 formed through the guide body 1301; a bone-facing surface on a first side of the guide body 1301; an obverse surface 1311 on a second side of the guide body 1301, opposite the bone-facing surface; a first guide arm 1341 projecting from the guide body 1301; a second guide arm 1342 projecting from the guide body 1301; a third guide arm 1343 projecting from the guide body 1301; a fourth guide arm 1344 projecting from the guide body 1301; a first guide arm bud 1351 coupled to the first guide arm 1341; a second guide arm bud 1352 coupled to the second guide arm 1342; a third guide arm bud 1353 coupled to the third guide arm 1343; a fourth guide arm bud 1354 coupled to the fourth guide arm 1344; one or more patient-specific surfaces, that may be analogous to the orthopedic guide 1100 shown in FIGS. 26C-27; and an offset guide handle 1370 coupled to the guide body 1301 with an offset guide handle shaft 1371 and one or more transverse guide handle holes 1372 formed therethrough.

However, it will be understood that in other embodiments the orthopedic guide 1300 may include any number of guide bodies, guide body projections, fixation guides/bores, guide holes/cannulas, guide arms, guide arm buds, patient-specific surfaces, handle structures, etc., and/or may incorporate any other feature, morphology, shape or orientation that is disclosed or contemplated herein with respect to any other guide.

In some embodiments, at least one guide feature associated with the guide body 1301 may comprise the guide body center punch hole 1310, which may be configured to guide an elongate surgical instrument (e.g., a guide wire, K-wire, drill bit, reference pin, etc.) into the bone 10 along a desired axis (e.g., a patient-specific axis that may be determined preoperatively, etc.). Once the elongate surgical instrument is inserted into the bone 10 along the desired patient-specific axis, the elongate surgical instrument may then be utilized to guide another surgical instrument (or an implant) relative to the bone 10 to a desired location onto or within the bone. For example, the elongate surgical instrument may be utilized to guide a cannulated reamer towards a surface of the bone 10 during a reaming operation to shape the glenoid cavity 6, etc.

In some embodiments, the orthopedic guide 1300 may include one or more markings which may be printed or otherwise formed on the orthopedic guide 1300 including, but not limited to: one or more orientation features configured to indicate an orientation of the orthopedic guide 1300 relative to the bone 10 of the patient, any instructions for use during the surgical procedure, any patient identifier information, any other labeling, etc.

In some embodiments, any portion(s) of the orthopedic guide 1300 may include one or more webbed/lattice structures to reduce an amount of material utilized during an additive manufacturing process.

In some embodiments, one or more of the guide arms may be configured to be selectively broken away from the guide body 1301 by the surgeon during the surgical procedure, as previously described herein.

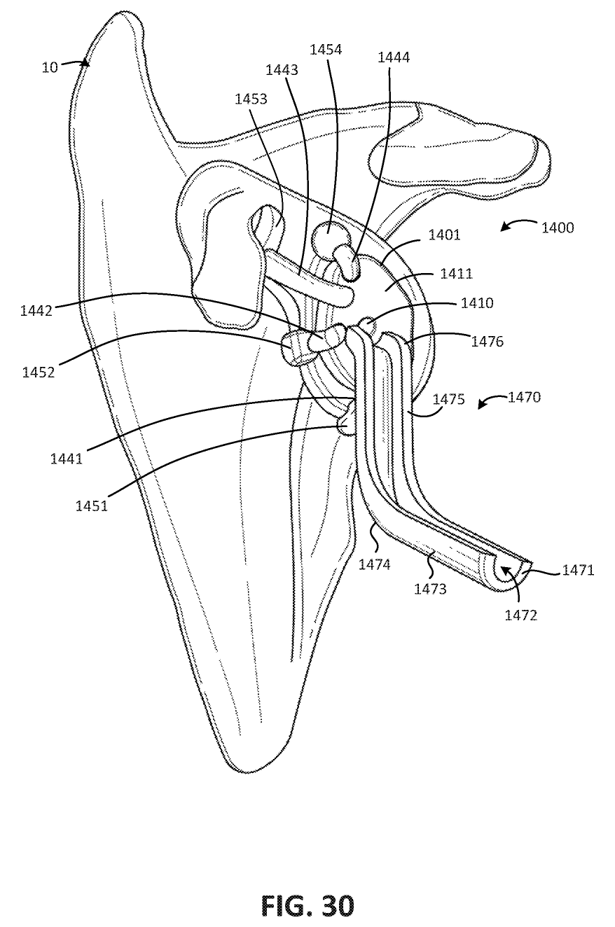

FIG. 30 illustrates an orthopedic guide 1400 engaged with a bone 10 (e.g., a glenoid cavity 6 on a scapula bone), according to another embodiment of the present disclosure.

In some embodiments, the orthopedic guide 1400 may generally include: a guide body 1401; a guide body center punch hole 1410 formed through the guide body 1401; a bone-facing surface on a first side of the guide body 1401; an obverse surface 1411 on a second side of the guide body 1401, opposite the bone-facing surface; a first guide arm 1441 projecting from the guide body 1401; a second guide arm 1442 projecting from the guide body 1401; a third guide arm 1443 projecting from the guide body 1401; a fourth guide arm 1444 projecting from the guide body 1401; a first guide arm bud 1451 coupled to the first guide arm 1441; a second guide arm bud 1452 coupled to the second guide arm 1442; a third guide arm bud 1453 coupled to the third guide arm 1443; a fourth guide arm bud 1454 coupled to the fourth guide arm 1444; one or more patient-specific surfaces, that may be analogous to the orthopedic guide 1100 shown in FIGS. 26C-27; and an alignment extension feature 1470 coupled to the obverse surface 1411 of the guide body 1401 and configured to couple with an alignment device (not shown) during the surgical procedure. The alignment extension feature 1470 may include a C-shaped channel 1471, with a hollow interior 1472, a proximal portion 1473, a proximal bend 1474, a distal portion 1475, and a distal bend 1476.

However, it will be understood that in other embodiments the orthopedic guide 1400 may include any number of guide bodies, guide body projections, fixation guides/bores, guide holes/cannulas, guide arms, guide arm buds, patient-specific surfaces, handle structures, alignment extension features, etc., and/or may incorporate any other feature, morphology, shape or orientation that is disclosed or contemplated herein with respect to any other guide.

In some embodiments, at least one guide feature associated with the guide body 1401 may comprise the guide body center punch hole 1410, which may be configured to guide an elongate surgical instrument (e.g., a guide wire, K-wire, drill bit, reference pin, etc.) into the bone 10 along a desired axis (e.g., a patient-specific axis that may be determined preoperatively, etc.). Once the elongate surgical instrument is inserted into the bone 10 along the desired patient-specific axis, the elongate surgical instrument may then be utilized to guide another surgical instrument (or an implant) relative to the bone 10 to a desired location onto or within the bone. For example, the elongate surgical instrument may be utilized to guide a cannulated reamer towards a surface of the bone 10 during a reaming operation to shape the glenoid cavity 6, etc.

In some embodiments, the orthopedic guide 1400 may include one or more markings which may be printed or otherwise formed on the orthopedic guide 1400 including, but not limited to: one or more orientation features configured to indicate an orientation of the orthopedic guide 1400 relative to the bone 10 of the patient, any instructions for use during the surgical procedure, any patient identifier information, any other labeling, etc.

In some embodiments, any portion(s) of the orthopedic guide 1400 may include one or more webbed/lattice structures to reduce an amount of material utilized during an additive manufacturing process.

In some embodiments, one or more of the guide arms may be configured to be selectively broken away from the guide body 1401 by the surgeon during the surgical procedure, as previously described herein.

Any procedures or methods disclosed herein may comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to "an embodiment" or "the embodiment" means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term "first" with respect to a feature or element does not necessarily imply the existence of a second or additional such feature or element. Elements recited in means-plus-function format are intended to be construed in accordance with 35 U.S.C. §112. It will be apparent to those having skill in the art that changes may be made to the details of the above-described embodiments without departing from the underlying principles set forth herein.

The phrases "connected to", "coupled to", "engaged with", and "in communication with" may refer to any form of interaction between two or more entities, including mechanical, electrical, magnetic, electromagnetic, fluid, and thermal interaction. Two components may be functionally coupled to each other even though they are not in direct contact with each other. The term "coupled" can include components that are coupled to each other via integral formation (e.g., integrally formed as a single piece, etc.), components that are removably and/or non-removably coupled with each other, components that are functionally coupled to each other through one or more intermediary components, etc. The term "abutting" refers to items that may be in direct physical contact with each other, although the items may not necessarily be attached together. The phrase "fluid communication" refers to two or more features that are connected such that a fluid within one feature is able to pass into another feature.

As defined herein the term "substantially" means within +/- 20% of a target value, measurement, or desired characteristic.

While specific embodiments and applications of the present disclosure have been illustrated and described, it is to be understood that the scope of the present disclosure is not limited to the precise configurations and components disclosed herein. Various modifications, changes, and variations which will be apparent to those skilled in the art may be made in the arrangement, operation, and details of the devices, systems, and methods disclosed herein.

1. An orthopedic guide comprising:
a guide body configured to couple with a humerus of a patient;
at least two guide features associated with the guide body that are configured to guide at least one surgical instrument relative to the humerus; and
a bone-facing surface associated with the guide body comprising a patient-specific surface formed thereon corresponding to an anatomical feature of the humerus,
wherein the patient-specific surface is configured to position the at least two guide features along at least two desired orientations relative to the humerus as the patient-specific surface engages the anatomical feature on the humerus.

2. The orthopedic guide of claim 1, wherein:
the at least one surgical instrument comprises:
a bone cutting instrument; and
the at least two guide features comprise:
a first cutting guide feature configured to guide the bone cutting instrument into the humerus along a first desired plane corresponding to a first implant surface of an implant that is configured to be installed on a first prepared surface of the humerus formed along the first desired plane; and
a second cutting guide feature configured to guide the bone cutting instrument into the humerus along a second desired plane corresponding to a second implant surface of the implant that is configured to be installed on a second prepared surface of the humerus that is formed along the second desired plane.

3. The orthopedic guide of claim 2, wherein at least one of the first desired plane and the second desired plane is not parallel to an anatomic neck plane of the humerus.

4. The orthopedic guide of claim 2, wherein:
the at least one surgical instrument further comprises:
an elongate surgical instrument; and
the at least two guide features further comprise:
a reference guide cannula configured to guide the elongate surgical instrument into the humerus along a desired axis.

5. The orthopedic guide of claim 1, further comprising at least one guide arm extending from the guide body and comprising the patient-specific surface formed thereon.

6. The orthopedic guide of claim 5, wherein the at least one guide arm is configured to selectively break away from the guide body.

7. The orthopedic guide of claim 1, wherein the patient-specific surface is at least partially formed by an additive manufacturing process.

8. An orthopedic guide comprising:
a guide body configured to couple with a humerus of a patient;
a cutting guide feature coupled with the guide body and configured to guide a cutting instrument along a desired cutting plane relative to the humerus that is not parallel to an anatomic neck plane of the humerus;
a first patient-specific surface formed on a first bone-facing surface of the guide body that corresponds to a first anatomical feature of the humerus; and
at least one guide arm coupled to and extending away from the guide body, the at least one guide arm comprising a second patient-specific surface formed on a second bone-facing surface of the at least one guide arm that corresponds to a second anatomical feature of the humerus;
wherein, the cutting guide feature is positioned along the desired cutting plane relative to the humerus when the first patient-specific surface engages the first anatomical feature of the humerus, and the second patient-specific surface engages the second anatomical feature of the humerus.

9. The orthopedic guide of claim 8, wherein the cutting guide feature comprises a cutting guide surface.

10. The orthopedic guide of claim 8, wherein the cutting guide feature comprises a cutting guide slot.

11. The orthopedic guide of claim 8, further comprising a guide body projection coupled to and extending away from the guide body.

12. The orthopedic guide of claim 8, wherein the at least one guide arm comprises a guide arm bud coupled thereto, the guide arm bud comprising the second patient-specific surface formed thereon.

13. The orthopedic guide of claim 8, wherein the at least one guide arm is configured to selectively break away from the guide body.

14. The orthopedic guide of claim 8, further comprising a fixation guide configured to receive a securement pin therethrough to couple the orthopedic guide to the humerus.

15. An orthopedic guide comprising:
a first guide body configured to couple with a bone of a patient comprising a first cutting guide feature;
a second guide body configured to couple with the bone of the patient comprising a second cutting guide feature;
at least one guide body bridge coupled intermediate the first guide body and the second guide body; and
at least one bone-facing surface associated with the orthopedic guide comprising at least one patient-specific surface formed thereon that corresponds to at least one anatomical feature of the bone,
wherein, when the at least one patient-specific surface is engaged with the at least one anatomical feature of the bone:
the first cutting guide feature is positioned at a first desired orientation relative to the bone; and
the second cutting guide feature is positioned at a second desired orientation relative to the bone.

16. The orthopedic guide of claim 15, wherein:
the first cutting guide feature comprises a first cutting surface encapsulated within a first cutting guide slot that is configured to guide a cutting surgical instrument into the bone along a first desired plane; and
the second cutting guide feature comprises a second cutting surface encapsulated within a second cutting guide slot that is configured to guide the cutting surgical instrument into the bone along a second desired plane.

17. The orthopedic guide of claim 15, wherein at least one of the first guide body and the second guide body comprise a lattice structure.

18. The orthopedic guide of claim 15, further comprising:
at least one guide arm extending from the orthopedic guide and comprising the at least one patient-specific surface formed thereon.

19. The orthopedic guide of claim 15, further comprising:
a reference guide cannula configured to guide an elongate surgical instrument into the bone along a desired axis.

20. The orthopedic guide of claim 15, wherein the bone comprises a humerus.
